(19) United States
(12) Patent Application Publication
(10) Pub. No.: US 2007/0293482 A1
(43) Pub. Date: Dec. 20, 2007

(54) Process for Preparing Benzodiazepines

(75) Inventors: Verity Dowdell, London (GB); Richard David Kelsey, London (GB); Malcolm Carter, London (GB); Elisa Henderson, London (GB)

Correspondence Address: LAHIVE & COCKFIELD, LLP, ONE POST OFFICE SQUARE, BOSTON, MA, 02109-2127, US

(73) Assignee: Novartis Pharmaceuticals Corporation, Cambridge, MA (US)

(21) Appl. No.: 10/593,665

(22) PCT Filed: Mar. 21, 2005
(86) PCT No.: PCT/GB05/01050

§ 371 Date: Aug. 2, 2007

(30) Foreign Application Priority Data
Mar. 19, 2004 (GB) 0406280.8
Mar. 19, 2004 (GB) 0406282.4
Oct. 21, 2004 (GB) 0423462.1

(51) Int. Cl. A61K 31/5513; A61P 31/12; C07D 243/24; C07D 243/26

(52) U.S. Cl. 514/221; 540/504; 540/509

(57) ABSTRACT

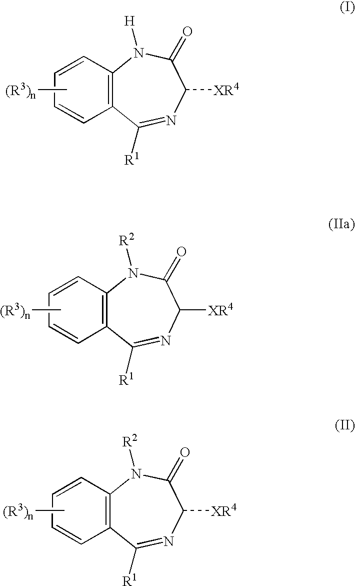

A process for producing a compound which is a benzodiazepine derivative of formula: (I) wherein: represents or R¹ represents C₁₋₆ alkyl, aryl or heteroaryl; each R³ is the same or different and represents halogen, hydroxy, C₁₋₆ alkyl, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆ alkyl)amino, nitro, cyano, —CO₂R′, —CONR′R″, —NH—CO—R′, —S(O)R′, —S(O)²R′, —NH—S(O)₂R′, —S(O)NR′R″ or —S(O)₂NR′R″, wherein each R′ and R″ is the same or different and represents hydrogen or C₁₋₆ alkyl; n is from 0 to 3; X represents —NH—, —N(C₁-C₆alkyl)-, —CO—, —CO—NR′—, —S(O)— or —S(O)₂—, wherein R′ is hydrogen or a C₁-C₆ alkyl group; and R⁴ represents hydrogen; or —CO—R₄′ or —CO—NH—R^{4 ′}, wherein R^{4 ′} is a C₁-C₆ alkyl, C₁-C₆ hydroxyalkyl, aryl, heteroaryl, carbocyclyl or heterocyclyl group, which group is substituted by a C₁-C₆ hydroxyalkyl, aryl, heteroaryl, carbocyclyl or heterocyclyl group or a —(C₁-C₄ alkyl)-X₁—(C₁-C₄ alkyl)-X₂—(C₁-C₄ alkyl) group, wherein X₁ represents —O—, —S— or —NR′—, wherein R′ represents H or a C₁-C₄ alkyl group and X₂ represents —CO—, —SO— or —SO₂—; or R^{4 ′}represents -A₁-Y-A₂, wherein: A₁ is an aryl, heteroaryl, carbocyclyl or heterocyclyl group; Y represents a direct bond or a C₁-C₄ alkylene, —SO₂—, —CO—, —O—, —S or —NR′—, wherein R′ is a C₁-C₆ alkyl group; and A₂ is an aryl, heteroaryl, carbocyclyl or heterocyclyl group; or R⁴ is a group selected from aryl-C(O)—C(O)—, heteroaryl-C(O)—C(O)—, carbocyclyl-C(O)—C(O)—, heterocyclyl-C(O)—C(O)— and -ZR⁵, wherein: Z represents —CO—, —S(O)— or —S(O)₂′; and R⁵ represents C₁₋₆ alkyl, hydroxy, C₁₋₆ alkoxy, C₁₋₆ alkylthio, aryl, heteroaryl, carbocyclyl, heterocyclyl, aryl-(C₁₋₆ alkyl)-, heteroaryl-(C₁₋₆ alkyl)-, carbocyclyl-(C₁₋₆ alkyl)-, heterocyclyl-(C₁₋₆ alkyl)-, aryl-(C₁₋₆ alkyl)-O—, heteroaryl-(C₁₋₆ alkyl)-O—, carbocyclyl-(C₁₋₆ alkyl)-O—, heterocyclyl-(C₁₋₆ alkyl)-O— or —NR′R″ wherein each R′ and R″ is the same or different and represents hydrogen, C₁₋₆ alkyl, carbocyclyl, heterocyclyl, aryl, heteroaryl, aryl-(C₁₋₆ alkyl)-, heteroaryl-(C₁₋₆ alkyl)-, carbocyclyl-(C₁₋₆ alkyl)- or heterocyclyl-(C₁₋₆ alkyl)-; or a pharmaceutically acceptable salt thereof; which process comprises: (a) subjecting a racemic benzodiazepine derivative of formula: (IIa): wherein R¹, R³, R⁴, n and X are as defined above, and R² represents an amino protecting group, to crystallisation induced dynamic resolution to yield a benzodiazepine derivative of formula (II): wherein, R¹, R², R³, R⁴, n and X are as defined above; and (b) deprotecting the benzodiazepine derivative of formula (II) as defined above to yield a benzodiazepine derivative of formula (I) or a pharmaceutically acceptable form thereof as defined above.

The present invention relates to process for producing a series of benzodiazepine derivatives which are active against Respiratory Syncytial Virus (RSV).

RSV is a major cause of respiratory illness in patients of all ages. In adults, it tends to cause mild cold symptoms. In school-aged children, it can cause a cold and bronchial cough. In infants and toddlers it can cause bronchiolitis (inflammation of the smaller airways of the lungs) or pneumonia. It has also been found to be a frequent cause of middle ear infections (otitis media) in pre-school children. RSV infection in the first year of life has been implicated in the development of asthma during childhood.

Particular benzodiazepine derivatives are known to be active against RSV. Research has shown that activity resides in one enantiomer of a racemic mixture. Previously known synthetic routes to the active isomers have proved unfeasible for scale-up to an industrial process because they contained several chromatographic separations. Conventional resolution of a racemic mixture of products involves discarding 50% of the material. Further, known syntheses involve capricious crystallisation to yield the desired product.

Reider et al, in J. Org. Chem. 1987, 52, 955-957, describe resolution of a benzodiazepine derivative, 3(RS)-amino-1,3-dihydro-1-methyl-5-phenyl-2H-1,4-benzodiazepin-2-one, using crystallisation induced dynamic resolution, in which the salt of the S-enantiomer favourably crystallised when stirring the racemic mixture with one equivalent (S)-CSA. This technique has been unsuccessfully applied to other benzodiazepine derivatives. Notably, resolution of 3(RS)-amino-1,3-dihydro-5-phenyl-2H-1,4-benzodiazepin-2-one has proved unsuccessful using this technique.

The present invention uses crystallisation induced dynamic resolution of benzodiazepine derivatives, in which the racemic precursor is converted to a single enantiomer in order to provide an improved yield synthesis of the RSV active enantiomer of a benzodiazepine derivative. Accordingly, the present invention provides a process for producing a compound which is a benzodiazepine derivative of formula (I): 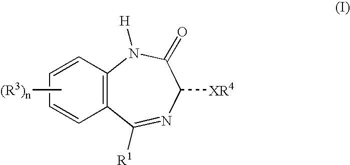
wherein:
[symbol] represents [symbol] or [symbol]
R¹ represents C₁₋₆ alkyl, aryl or heteroaryl;
each R³ is the same or different and represents halogen, hydroxy, C₁₋₆ alkyl, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆ alkyl)amino, nitro, cyano, —CO₂R′, —CONR′R″, —NH—CO—R′, —S(O)R′, —S(O)₂R′, —NH—S(O)₂R′, —S(O)NR′R″ or —S(O)₂NR′R″, wherein each R′ and R″ is the same or different and represents hydrogen or C₁₋₆ alkyl;
n is from 0 to 3;
X represents —NH—, —N(C₁-C₆ alkyl)-, —CO—, —CO—NR′—, —S(O)— or —S(O)₂—, wherein R′ is hydrogen or a C₁-C₆ alkyl group; and
R⁴ represents hydrogen; or —CO—R₄′ or —CO—NH—R₄′, wherein R₄′ is a C₁-C₆ alkyl, C₁-C₆ hydroxyalkyl, aryl, heteroaryl, carbocyclyl or heterocyclyl group, which group is substituted by a C₁-C₆ hydroxyalkyl, aryl, heteroaryl, carbocyclyl or heterocyclyl group or a —(C₁-C₄ alkyl)-X₁—(C₁-C₄ alkyl)-X₂—(C₁-C₄ alkyl) group, wherein X₁ represents —O—, —S— or —NR′—, wherein R′ represents H or a C₁-C₄ alkyl group and X₂ represents —CO—, —SO— or —SO₂′; or R₄′ represents -A₁-Y-A₂, wherein:

A₁ is an aryl, heteroaryl, carbocyclyl or heterocyclyl group;

Y represents a direct bond or a C₁-C₄ alkylene, —SO₂—, —CO—, —O—, —S or —NR′—,

wherein R′is a C₁-C₆ alkyl group; and

A₂ is an aryl, heteroaryl, carbocyclyl or heterocyclyl group;

or R⁴ is a group selected from aryl-C(O)—C(O)—, heteroaryl-C(O)—C(O)—, carbocyclyl-C(O)—C(O)—, heterocyclyl-C(O)—C(O)— and -ZR⁵, wherein:

Z represents —CO—, —S(O)— or —S(O)₂—; and

R⁵ represents C₁₋₆ alkyl, hydroxy, C₁₋₆ alkoxy, C₁₋₆ alkylthio, aryl, heteroaryl, carbocyclyl, heterocyclyl, aryl-(C₁₋₆ alkyl)-, heteroaryl-(C₁₋₆ alkyl)-, carbocyclyl-(C₁₋₆ alkyl)-, heterocyclyl-(C₁₋₆ alkyl)-, aryl-(C₁₋₆ alkyl)-O—, heteroaryl-(C₁₋₆ alkyl)-O—, carbocyclyl-(C₁₋₆ alkyl)-O—, heterocyclyl-(C₁₋₆ alkyl)-O— or —NR′R″ wherein each R′ and R″ is the same or different and represents hydrogen, C₁₋₆ alkyl, carbocyclyl, heterocyclyl, aryl, heteroaryl, aryl-(C₁₋₆ alkyl)-, heteroaryl-(C₁₋₆ alkyl)-, carbocyclyl-(C₁₋₆ alkyl)- or heterocyclyl-(C₁₋₆ alkyl)-;

or a pharmaceutically acceptable salt thereof; which process comprises:

(a) subjecting a racemic benzodiazepine derivative of formula (IIa): 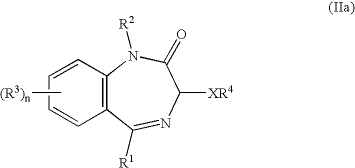
   wherein R¹, R³, R⁴, n and X are as defined above, and
R² represents an amino protecting group, to crystallisation induced dynamic resolution to yield a benzodiazepine derivative of formula (II): 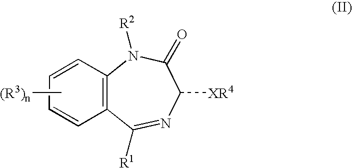
   wherein [symbol] R¹, R², R³, R⁴, n and X are as defined above; and
(b) deprotecting the benzodiazepine derivative of formula(II) as defined above to yield a benzodiazepine derivative of formula (I) or a pharmaceutically acceptable salt thereof as defined above.

In one aspect of the process of the present invention the benzodiazepine of formula (I) as the following structure (I′): 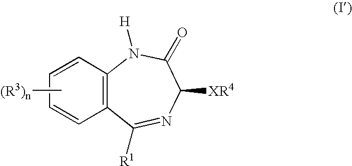
wherein R¹, R³, R⁴, n and X are as defined above.

R⁴ can also represent hydrogen; a group selected from C₁-C₆ alkyl, C₁-C₆ hydroxyalkyl, aryl, heteroaryl, carbocyclyl and heterocyclyl, which group is substituted by a C₁-C₆ hydroxyalkyl, aryl, heteroaryl, carbocyclyl or heterocyclyl group or a —(C₁-C₄ alkyl)-X₁—(C₁-C₄ alkyl)-X₂—(C₁-C₄ alkyl) group, wherein X₁ represents —O—, -S— or —NR′—, wherein R′ represents H or a C₁-C₄ alkyl group and X₂ represents —CO—, —SO— or —SO₂—; -A₁-Y-A₂, wherein:

A₁ is an aryl, heteroaryl, carbocyclyl or heterocyclyl group;

Y represents a direct bond or a C₁-C₄ alkylene, —SO₂—, —CO—, —O—, —S or —NR′—,

wherein R′is a C₁-C₆ alkyl group; and

A₂ is an aryl, heteroaryl, carbocyclyl or heterocyclyl group;

or a group selected from aryl-C(O)—C(O)—, heteroaryl-C(O)—C(O)—, carbocyclyl-C(O)—C(O)—, heterocyclyl-C(O)—C(O)— and -ZR⁵, wherein:

Z represents —CO—, —S(O)— or —S(O)₂—; and

R⁵ represents C₁₋₆ alkyl, hydroxy, C₁₋₆ alkoxy, C₁₋₆ alkylthio, aryl, heteroaryl, carbocyclyl, heterocyclyl, aryl-(C₁₋₆ alkyl)-, heteroaryl-(C₁₋₆ alkyl)-, carbocyclyl-(C₁₋₆ alkyl)-, heterocyclyl-(C₁₋₆ alkyl)-, aryl-(C₁₋₆ alkyl)-O—, heteroaryl-(C₁₋₆ alkyl)-O—, carbocyclyl-(C₁₋₆ alkyl)-O—, heterocyclyl-(C₁₋₆ alkyl)-O— or —NR′R″ wherein each R′ and R″ is the same or different and represents hydrogen, C₁₋₆ alkyl, carbocyclyl, heterocyclyl, aryl, heteroaryl, aryl-(C₁₋₆ alkyl)-, heteroaryl-(C₁₋₆ alkyl)-, carbocyclyl-(C₁₋₆ alkyl)- or heterocyclyl-(C₁₋₆ alkyl)-.

Crystallisation induced dynamic resolution (CIDR) is an example of dynamic kinetic resolution (DKR). Alternative dynamic kinetic resolution techniques may be applied in the process of the present invention. CIDR utilises an equilibrium between two enantiomers and the different affinities of the two enantiomers of a given compound for an optically active partner organic acid. One enantiomer (the desired enantiomer) preferentially crystallises with the partner organic acid to yield a salt of that enantiomer, leaving the other enantiomer in solution. The desired enantiomer is then recovered by converting the said salt into the corresponding free compound by conventional techniques.

The racemate must be held in conditions that allow spontaneous racemisation. Thus when one enantiomer crystallises, the equilibrium is displaced and returns by racemisation of the remaining enantiomers. Separation of the product enantiomer from the unwanted enantiomer is dynamic, with the unwanted enatiomer being converted to the desired enantiomer. This technique leads to theoretical yields of 100% of the desired enantiomer, compared with a theoretical yield of 50% using conventional techniques.

Suitable optically active organic acids for use in CIDR include (S)-tartaric acid, (S)-CSA ((S)-camphosulphonic acid), N-Boc-(S)-phenylalanine (N-tertiarybutoxycarbonyl-(S)-phenylalanine), (S)-3-phenyllactic acid, (S)-mandelic acid, (S)-lactic acid, (R)-2,2-dimethyl-5-oxo-1,3-dioxolane-4-acetic acid. Typically the organic acid used in the present process is (S)-CSA or N-Boc-(S)-phenylalanine.

Suitable solvents for use in CIDR include toluene, diethylether (Et₂O), dichloromethane (DCM), ethanol (EtOH), ethylacetate (EtOAc), diisopropylether (ⁱPr₂O), isopropylacetate (ⁱPrOAc) and acetonitrile (MeCN).

Racemisation is aided by the addition of a racemisation promoting agent to the racemate. Suitable racemisation promoting agents when X in formula (IIa) is —NH— or —N(C₁-C₆ alkyl)- include those that reversibly convert the said amine to an imine. Examples of such racemisation promoting agents include aldehydes, such as aromatic aldehydes. Typically 3,5-dichlorosalicylaldehyde is used.

The presence of water in the CIDR reaction mixture is known to aid crystallisation induced dynamic resolution. Typically an amount of water is present in the process of the present invention. Preferably from 0.01 to 5 reaction equivalents of water are present, more preferably from 0.05 to 1 reaction equivalents of water are present.

A seed crystal of the desired salt is typically added to the racemate, in order to aid initiation of crystallisation.

The racemate may be subjected to ultrasonic treatment. Applying an ultrasonic frequency to the racemate promotes homogenisation of the solution.

R² is any amino protecting group known in the art. Examples of such groups are, for instance, described in “Protective Groups for Organic Chemistry”, Third Edition, T. W. Greene and P. G. M. Wuts, John Wiley and Sons, 1999. An amino group can be protected as an amide such as N-methylacetamide, a thioamide such as N-methylacetathioamide, a carbamate, a thiocarbamate, an imide, urea, thiourea or guanidine. Typical examples of amino protecting groups thus include phthalimidoyl, tetrachlorophthalimidoyl, dithiasuccinoyl and trifluoroacetyl groups; methoxycarbonyl, ethoxycarbonyl, t-butyloxycarbonyl, benzyloxycarbonyl, 9-fluorenylmethyloxycarbonyl, 2-(trimethylsilyl)ethoxycarbonyl and 2,2,2-trichloroethoxycarbonyl groups; and methylthiocarbonyl, ethylthiocarbonyl, t-butylthiocarbonyl, benzylthiocarbonyl, 9-fluorenylmethylthiocarbonyl, 2-(trimethylsilyl)ethylthiocarbonyl and 2,2,2-trichloroethylthiocarbonyl groups. Other examples of amino protecting groups include sulfonyl groups, for instance 2-(trimethylsilyl)ethylsulfonyl; alkyl and aryl groups as defined above, for instance methyl, ethyl, n-propyl, n-butyl, benzyl, diphenylmethyl, trityl and 9-phenylfluoromethyl groups. An amino group may also be protected as an imine derivative, for instance an imine with a bis(methylthio)methylene or diphenylmethylene group; or as a hydroxylamine, for instance N-t-butyl hydroxylamine or biphenyl ether N-formyl-hydroxylamine.

Typically the protecting group R² is a group —(CH₂)ₘ—R′, wherein m is 0 or an integer of from 1 to 3 and R′ is a group —O—(C₁₋₆ alkyl), —C(O)O—(C₁₋₆ alkyl), —OC(O)—(C₁₋₆ alkyl), aryl, heteroaryl, carbocyclyl or heterocyclyl.

The deprotection step (b) involves replacement of the moiety R² with a hydrogen atom. This may be achieved by any suitable means. The means of deprotection employed depend on the nature of the R² and the other substituents R¹, R³, R⁴ and X. The reagents for deprotection are selected for their suitability at selectively removing R₂ without adversely affecting the rest of the compound. Deprotection conditions may be either acidic or basic. For instance deprotection may be carried out in the presence of a Lewis Acid, such as aluminium chloride, boron trifluoride, titanium tetrachloride, or the like. Typical reagents for deprotection include ceric ammonium nitrate (CAN), trifluoroacetic acid (TFA), hydrogenbromide/acetic acid, aluminium trichloride/anisole (AlCl₃/PhOMe), AlClₛ thioanisole, 2,3-dichloro-5,6-dicyano-1,4-benzoquinone (DDQ) and sodium/ammonia (Na/NH₃). AlCl₃ is preferred. These reactions are carried out in a suitable inert solvent, such as anisole or thioanisole. Typically the solvent has cationic scavenging properties. Reaction temperatures may range from −20° C. to 150° C., but are typically between room temperature and 0° C.

As used herein, a C₁₋₆ alkyl group or moiety is a linear or branched alkyl group or moiety containing from 1 to 6 carbon atoms, such as a C₁₋₄ alkyl group or moiety. Examples of C₁₋₄ alkyl groups and moieties include methyl, ethyl, n-propyl, i-propyl, n-butyl, i-butyl and t-butyl. For the avoidance of doubt, where two alkyl moieties are present in a group, the alkyl moieties may be the same or different.

As used herein, a hydroxyalkyl group is typically a said alkyl group that is substituted by one or more hydroxy groups. Typically, it is substituted by one, two or three hydroxy groups. Preferably, it is substituted by a single hydroxy group. Preferred hydroxyalkyl groups are (monohydroxy)ethyl groups and CH₂—OH.

As used herein, an acyl group is a C₂₋₇ acyl group, for example a group —CO—R, wherein R is a said C₁₋₆ alkyl group.

As used herein, an aryl group is typically a C₆₋₁₀ aryl group such as phenyl or naphthyl. Phenyl is preferred. An aryl group may be unsubstituted or substituted at any position. Typically, it carries 0, 1, 2 or 3 substituents.

Suitable substitutents on an aryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl. Examples of suitable substitutents on an aryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —NH—S(O)₂R′ or —NH—CO—R′, wherein each R′ and R″ is the same or different and represents hydrogen or C₁₋₆ alkyl.

Preferred substituents on an aryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆ alkyl)amino, nitro, cyano, —CO₂R′, —S(O)R′, —S(O)₂R′ and —S(O)₂NR′R″, wherein each R′ and R″ is the same or different and represents hydrogen or C₁₋₄ alkyl. Examples of preferred substituents on an aryl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano.

Particularly preferred substituents include fluorine, chlorine, bromine, iodine, C₁₋₄ alkyl, C₂₋₄ acyl, hydroxy, C₁₋₄ alkoxy, C₁₋₄ alkylthio, C₁₋₄ haloalkyl, C₁₋₄ haloalkoxy, amino, mono(C₁₋₄ alkyl)amino, di(C₁₋₄ alkyl)amino, nitro, —CO₂R′, —S(O)₂R′ and —S(O)₂NH₂, wherein R′ represents C₁₋₂ alkyl. Examples of particularly preferred substituents include fluorine, chlorine, bromine, cyano, C₁₋₄ alkyl, C₁₋₄ alkoxy, C₁₋₄ haloalkyl and nitro, for instance methyl, ethyl, methoxy and ethoxy.

As used herein, references to an aryl group include fused ring systems in which an aryl group is fused to a monocyclic carbocyclyl, heterocyclyl or heteroaryl group or to a monocyclic carbocyclyl, heterocyclyl or heteroaryl group which is fused to a phenyl ring. Typically, said fused ring systems are systems in which an aryl group is fused to a monocyclic carbocyclyl, heterocyclyl or heteroaryl group.

Preferred such ring systems are those wherein an aryl group is fused to a fused group which is a monocyclic heterocyclyl or heteroaryl group or to a monocyclic carbocyclic group fused to a phenyl ring, in particular those wherein an aryl group is fused to a heterocyclyl or heteroaryl group. Examples of such fused ring systems are groups in which a phenyl ring is fused to a thienyl group or to a tetrahydrofuranyl group to form a benzothienyl or dihydrobenzofuranyl group. Further examples of such fused rings are groups in which a phenyl ring is fused to a dioxanyl group, a pyrrolyl group or a 2,3-dihydroinden-1-one group to form a benzodioxinyl, indolyl or a 9H-fluoren-9-one group.

As used herein, a carbocyclyl group is a non-aromatic saturated or unsaturated monocyclic hydrocarbon ring, typically having from 3 to 6 carbon atoms. Preferably it is a saturated hydrocarbon ring (i.e. a cycloalkyl group) having from 3 to 6 carbon atoms. Examples include cyclopropyl, cyclobutyl, cyclopentyl and cyclohexyl. It is preferably cyclopropyl, cyclopentyl or cyclohexyl, most preferably cyclopropyl. A cycloalkyl group may be unsubstituted or substituted at any position. Typically, it carries 0, 1, 2 or 3 substituents.

Suitable substitutents on a carbocyclyl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, oxo, —CO₂R′, —CONR′R″, —S(O)R′, —S(O)₂R′, —S(O)NR′R″, —S(O)₂NR′R″, —NH—S(O)₂R′ or —NH—CO′R′, wherein each R′ and R″ is the same or different and represents hydrogen or C₁₋₆ alkyl. Examples of suitable substitutents on a carbocyclyl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —NH—S(O)₂R′ or —NH—CO—R′, wherein each R′ and R″ is the same or different and represents hydrogen or C₁₋₆ alkyl.

Preferred substituents on a carbocyclyl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro, cyano and oxo. Examples of preferred substituents on an carbocyclyl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano. Particularly preferred substituents include fluorine, chlorine, bromine, C₁₋₄ alkyl, C₁₋₄ alkoxy, C₁₋₄ haloalkyl, nitro and oxo. Examples of particularly preferred substituents include fluorine, chlorine, bromine, C₁₋₄ alkyl, C₁₋₄ alkoxy, C₁₋₄ haloalkyl and nitro. Further examples of particularly preferred substituents include fluorine, C₁₋₄ alkyl, C₁₋₄ alkoxy, C₁₋₄ haloalkyl and nitro.

As used herein, a heterocyclyl group is a non-aromatic saturated or unsaturated carbocyclic ring typically having from 5 to 10 carbon atoms, in which one or more, for example 1, 2 or 3, of the carbon atoms is replaced by a heteroatom selected from N, O and S. Saturated heterocyclyl groups are preferred. Examples include tetrahydrofuranyl, tetrahydrothienyl, pyrrolidinyl, imidazolidinyl, pyrazolidinyl, dioxolanyl, thiazolidinyl, tetrahydropyranyl, piperidinyl, dioxanyl, piperazinyl, morpholinyl, thiomorpholinyl and thioxanyl. Further examples include dithiolanyl, oxazolidinyl, tetrahydrothiopyranyl and dithianyl. Piperazinyl, piperidinyl, thiomorpholinyl, imidazolidinyl and morpholinyl are preferred.

As used herein, references to a heterocyclyl group include fused ring systems in which a heterocyclyl group is fused to a phenyl group. Preferred such fused ring systems are those wherein a 5- to 6-membered heterocyclyl group is fused to a phenyl group. An example of such a fused ring system is a group wherein a 1H-imidazol-2(3H)-onyl group or a imidazolidin-2-onyl group is fused to a phenyl ring to form a 1H-benzo[d]imidazol-2(3H)-onyl group. Most preferably, however, a heterocyclyl group is monocyclic.

A heterocyclic group may be unsubstituted or substituted at any position. Typically, it carries 0, 1 or 2 substituents.

Suitable substitutents on a heterocyclyl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbomyl, amino, mono(C₁₋₆ alkyl)amino, di(C₁₋₆ alkyl)amino, oxo, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl. Examples of suitable substitutents on a heterocyclyl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbomyl, amino, mono(C₁₋₆ alkyl)amino, di(C₁₋₆ alkyl)amino, —CO₂R′, —CONR′R″, —S(O)R′, —S(O)₂R′, —S(O)NR′R″, —NH—S(O)₂R′ or —NH—CO—R′, wherein each R′ and R″ is the same or different and represents hydrogen or C₁₋₆ alkyl.

Preferred substituents on a heterocyclyl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro, cyano and oxo. Examples of preferred substituents on a heterocyclyl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano. Particularly preferred substituents include fluorine, chlorine, bromine, C₁₋₄ alkyl, C₁₋₄ alkoxy, C₁₋₄ haloalkyl, nitro and oxo. Examples of particularly preferred substituents include fluorine, chlorine, bromine, C₁₋₄ alkyl, C₁₋₄ alkoxy, C₁₋₄ haloalkyl and nitro. Further examples of particularly preferred substituents include fluorine, C₁₋₄ alkyl, C₁₋₄ alkoxy, C₁₋₄ haloalkyl and nitro. Most preferably, a heterocyclyl group is unsubstituted or substituted by one or two C₁₋₂ alkyl or oxo groups. An example of a substituted heterocyclic group is S,S-dioxo-thiomorpholino.

As used herein, a halogen is typically chlorine, fluorine, bromine or iodine. It is preferably chlorine, fluorine or bromine. It is more preferably chlorine or fluorine.

As used herein, an alkoxy group is typically a said alkyl group attached to an oxygen atom. An alkylthio group is typically a said alkyl group attached to a thio group. A haloalkyl or haloalkoxy group is typically a said alkyl or alkoxy group substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom, for example chlorine or fluorine. Particularly preferred haloalkyl groups are —CF₃ and —CCl₃. Particularly preferred haloalkoxy groups are —OCF₃ and —OCCl₃.

As used herein, a heteroaryl group is typically a 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, pyrazolidinyl, pyrrolyl, oxadiazolyl, isoxazolyl, thiadiazolyl, thiazolyl, imidazolyl and pyrazolyl groups. Further examples include oxazolyl and isothiazolyl. Preferred heteroaryl groups are pyridyl, thienyl, oxazolyl, isoxazolyl, furanyl and pyrazolyl. Examples of preferred heteroaryl groups are pyridyl, thienyl, isoxazolyl and furanyl. In the definition of R⁴ above, heteroaryl wherever it appears is typically other than furanyl. More particularly, when R′ or R″ in the definition of R⁵ is or comprises heteroaryl, the heteroaryl group is typically other than furanyl.

As used herein, references to a heteroaryl group include fused ring systems in which a heteroaryl group is fused to a phenyl group or to a monocyclic heterocyclyl group. Preferred such fused ring systems are those wherein a 5- to 6-membered heteroaryl group is fused to a phenyl group. Examples of such fused ring systems are benzofuranyl, benzothiophenyl, indolyl, benzimidazolyl, benzoxazolyl, quinolinyl, quinazolinyl, isoquinolinyl and 1H-imidazol[4,5-b]pyridin-2(3H)-one moieties. Most preferably a heterocyclyl group is monocyclic or fused to a 1H-imidazol[4,5-b]pyridin-2(3H)-one moiety.

A heteroaryl group may be unsubstituted or substituted at any position. Typically, it carries 0, 1, 2 or 3 substituents.

Suitable substitutents on a heteroaryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl. Examples of suitable substitutents on a heteroaryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —NH—S(O)₂R′ or —NH—CO—R′, wherein each R′ and R″ is the same or different and represents hydrogen or C₁₋₆ alkyl.

Preferred substituents on a heteroaryl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano. Particularly preferred substituents include fluorine, chlorine, bromine, C₁₋₄ alkyl, C₁₋₄ alkoxy, C₁₋₄ haloalkyl and nitro. Further preferred substituents include fluorine, chlorine, bromine, C₁₋₂ alkyl, C₁₋₂ haloalkyl and di(C₁₋₂ alkyl)amino.

As used herein, references to a heteroaryl group include fused ring systems in which a heteroaryl group is fused to a monocyclic said aryl, carbocyclyl or heterocyclyl group, or to a further heteroaryl group. Preferred such ring systems are those wherein a heteroaryl group is fused to an aryl group, for example a phenyl group. An example of such a fused ring system is a group wherein a thienyl group is fused to a phenyl ring to form a benzothienyl group. A further example of such a fused ring system is a group wherein a furanyl group is fused to a phenyl ring to form a benzofuranyl group.

When R¹ is an aryl or heteroaryl group it is typically unsubstituted or substituted by one, two or three substituents selected from halogen, C₁₋₆ alkyl, C₁₋₆ alkoxy, C₁₋₆ alkylthio, C₁₋₆ haloalkyl or C₁₋₆ haloalkoxy. Preferably, it is unsubstituted or substituted by one or two substituents selected from fluorine, chlorine, bromine, C₁₋₄ alkyl, C₁₋₄ alkoxy, C₁₋₄ alkylthio, C₁₋₄ haloalkyl or C₁₋₄ haloalkoxy. More preferably, it is unsubstituted or substituted by a single fluorine, chlorine, C₁₋₂ alkyl, C₁₋₂ alkoxy, C₁₋₂ alkylthio, C₁₋₂ haloalkyl or C₁₋₂ haloalkoxy substituent.

Typically, R¹ is C₁₋₆ alkyl or aryl. Preferably, R¹ is C₁₋₂ alkyl or aryl. More preferably, R¹ is C₁₋₂ alkyl or phenyl. More preferably, R¹ is phenyl.

Typically R² is (CH₂)ₘR′, wherein m is 1 or 2, R′ is a group O—(C₁₋₆ alkyl), —C(O)O—(C₁₋₆ alkyl) —OC(O)—(C₁₋₆ alkyl), aryl, heteroryl, carbocyclyl or heterocycyl. Preferably R² is a group —O—(C₁₋₄ alkyl), —C(O)O—(C₁₋₄ alkyl), —OC(O)—(C₁₋₄ alkyl) or aryl, which aryl is preferably phenyl, more preferably phenyl substituted by from 1 to 3 C₁₋₄ alkoxy groups. Examples of R² are paramethoxybenxyl, benzyl, 2,4,6-trimethoxybenzyl, 2,4-dihydroxybenzyl, pivalaloyloxymethyl, acetyl, methoxymethyl or tertiarybutoxy carbonyloxy.

Typically, R³ is halogen, hydroxy, C₁₋₄ alkyl, C₁₋₄ alkoxy, C₁₋₄ alkylthio, C₁₋₄ haloalkyl, C₁₋₄ haloalkoxy, amino, mono(C₁₋₄ alkyl)amino or di(C₁₋₄ alkyl)amino. Preferably, R³ is fluorine, chlorine, bromine, C₁₋₂ alkyl, C₁₋₂ alkoxy, C₁₋₂ alkylthio, C₁₋₂ haloalkyl, C₁₋₂ haloalkoxy, amino, mono(C₁₋₂ alkyl)amino or di(C₁₋₂ alkyl)amino. More preferably, R³ is methyl, trifluoromethyl, fluorine, chlorine or bromine. Most preferably, R³ is methyl or chlorine. An example of a most preferred group is when R³ is chlorine.

Typically, n is 0, 1 or 2. Preferably, n is 0 or 1.

Typically X is —NH—, —N(C₁₋₆ alkyl)- or —CO—. Preferably X is —NH—.

When R⁴ is a heterocyclyl group, it is typically attached via a carbon atom. Typically, R⁴ is C₁₋₆ alkyl, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)-, aryl-C(O)—C(O)—, heteroaryl-C(O)—C(O)— or -ZR⁶. Examples of typical R⁴ groups are those wherein R⁴ is C₁₋₆ alkyl, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)- or -ZR⁵.

Preferably, R⁴ is C₁₋₄ alkyl, aryl, for example phenyl and dihydrobenzofuranyl, heteroaryl, for example thienyl, isoxazolyl, pyridyl and benzothienyl, carbocyclyl, for example cyclopentyl and cyclohexyl, heterocyclyl, for example piperidinyl, morpholinyl and piperazinyl, phenyl-(C₁₋₂ alkyl)-, for example benzyl, heteroaryl-(C₁₋₂ alkyl)-, phenyl-C(O)—C(O)—, heteroaryl-C(O)—C(O)— or -ZR⁵. Examples of preferred R⁴ groups are those wherein R⁵ is C₁₋₄ alkyl, aryl, for example phenyl and dihydrobenzofuranyl, heteroaryl, for example thienyl, isoxazolyl, pyridyl and benzothienyl, carbocyclyl, for example cyclopentyl and cyclohexyl, heterocyclyl, for example piperidinyl, morpholinyl and piperazinyl, phenyl-(C₁₋₂ alkyl)-, for example benzyl, heteroaryl-(C₁₋₂ alkyl)- or -ZR⁵.

More preferably, R⁴ is C₁₋₄ alkyl, phenyl, thienyl, isoxazolyl, pyridyl, cyclopentyl, cyclohexyl, benzothienyl, dihydrobenzofuranyl, phenyl-CH₂—, phenyl-C(O)—C(O)—, thienyl-C(O)—C(O)— or -ZR⁵. Examples of more preferred R⁴ groups are those wherein R⁴ is C₁₋₄ alkyl, phenyl, thienyl, isoxazolyl, pyridyl, cyclopentyl, cyclohexyl, benzothienyl, dihydrobenzofuranyl, phenyl-CH₂— or -ZR⁵.

Most preferably, R⁴ is phenyl-CH₂—, —C(O)—C(O)-thienyl or -ZR⁵. Examples of most preferred R⁴ groups are those wherein R⁴ is phenyl-CH₂—, or -ZR⁵.

Typically, Z is —CO—, —S(O)— or —S(O)₂—. Preferably Z is —CO— or —S(O)₂—.

When R⁵ is a group —NR′R″ and either R′ or R″ includes an aryl, heteroaryl, carbocyclyl or heterocyclyl moiety it is typically unsubstituted or substituted by 1, 2 or 3 substituents selected from halogen, C₁₋₆ alkyl, C₁₋₆ alkoxy, C₁₋₆ alkylthio, C₁₋₆ haloalkyl, C₁₋₆ haloalkoxy, nitro and cyano. Preferably, the aryl, heteroaryl, carbocyclyl or heterocyclyl moiety is unsubstituted or substituted by 1 or 2 substituents selected from fluorine, chlorine, bromine, C₁₋₄ alkyl, C₁₋₄ alkoxy, C₁₋₄ alkylthio, C₁₋₄ haloalkyl, C₁₋₄ haloalkoxy and nitro. An example of preferred substitution is when the aryl, heteroaryl, carbocyclyl or heterocyclyl moiety is unsubstituted or substituted by 1 or 2 substituents selected from fluorine, chlorine, bromine, C₁₋₄ alkyl, C₁₋₄ alkoxy, C₁₋₄ haloalkyl and nitro. More preferably, the aryl, heteroaryl, carbocyclyl or heterocyclyl moiety is unsubstituted or substituted by one or two substituents selected from fluorine, chlorine, bromine, C₁₋₂ alkyl, C₁₋₂ alkoxy, C₁₋₂ alkylthio, C₁₋₂ haloalkyl and nitro. An example of more preferred substitution is when the aryl, heteroaryl, carbocyclyl or heterocyclyl moiety is unsubstituted or substituted by a single fluoro, chloro, methyl, methoxy or nitro substituent. When R′ or R″ is a heteroaryl or heterocyclyl group, it is attached via a carbon atom.

Typically, R′ and R″ are not both hydrogen. Typically, each R′ and R″ is the same or different and represents hydrogen, C₁₋₄ alkyl, aryl, heteroaryl, carbocyclyl, aryl-(C₁₋₄ alkyl)- or heteroaryl-(C₁₋₄ alkyl)-. Examples of typical R′ and R″ groups are those wherein each R′ and R″ is the same or different and represents hydrogen, C₁₋₄ alkyl, phenyl, heteroaryl, for example thienyl, carbocyclyl, for example cyclohexyl or cyclopentyl, or phenyl-(C₁₋₄ alkyl)-. Further examples of typical R′ and R″ groups are those wherein each R′ and R″ is the same or different and represents hydrogen, C₁₋₄ alkyl, phenyl, thienyl, cyclohexyl, cyclopentyl or phenyl-(CH₂)—. Preferably, each R′ and R″ is the same or different and represents hydrogen, C₁₋₄ alkyl, phenyl, phenyl-CH₂—, cyclohexyl or cyclopentyl. More preferably, one of R′ and R″ represents hydrogen. Most preferably, one of R′ and R″ is hydrogen and the other is C₁₋₄ alkyl, phenyl, phenyl-CH₂—, cyclohexyl or cyclopentyl. As an additional preference, one of R′ and R″ is hydrogen and the other is C₁₋₄ alkyl, phenyl, thienyl or phenyl-CH₂—.

Typically, R⁵ is C₁₋₆ alkyl, hydroxy, C₁₋₆ alkoxy, C₁₋₆ alkylthio, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)-, aryl-(C₁₋₄ hydroxyalkyl)-, heteroaryl-(C₁₋₄ hydroxyalkyl)-, carbocyclyl-(C₁₋₄ hydroxyalkyl)-, heterocyclyl-(C₁₋₄ hydroxyalkyl)-, aryl-(C₁₋₄ alkyl)-O—, heteroaryl-(C₁₋₄ alkyl)-O—, carbocyclyl-(C₁₋₄ alkyl)-O—, heterocyclyl-(C₁₋₄ alkyl)-O— or —NR′R″ wherein R′ and R″ are as defined above. Examples of typical R⁵ groups are those wherein R⁵ is C₁₋₆ alkyl, hydroxy, C₁₋₆ alkoxy, C₁₋₆ alkylthio, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)- or —NR′R″ wherein R′ and R″ are as defined above.

Preferably, R⁵ is C₁₋₆ alkyl, C₁₋₆ alkoxy, C₁₋₆ alkylthio, aryl, for example phenyl, naphthyl, dihydrobenzofuranyl, benzodioxinyl, 9H-fluoren-9-onyl and indolyl, heteroaryl, for example thienyl, furanyl, oxazolyl, isoxazolyl, pyrazolyl, pyridyl, benzothienyl and benzofuranyl, carbocyclyl, for example cyclopentyl and cyclohexyl, heterocyclyl, for example piperazinyl, piperidinyl, morpholinyl and 1H-benzo[d]imidazol-2(3H)-onyl, phenyl-(C₁₋₂ alkyl)-, phenyl-(C₁₋₂ alkyl)-O—, phenyl-(C₁₋₂ hydroxyalkyl)-, heteroaryl-(C₁₋₂ hydroxyalkyl)-, heteroaryl-(C₁₋₂ alkyl)- or —NR′R″ wherein R′ and R″ are as defined above. Examples of preferred R⁵ groups are those wherein R⁵ is C₁₋₄ alkyl, aryl, for example phenyl and dihydrobenzofuranyl, heteroaryl, for example thienyl, furanyl, isoxazolyl, pyridyl and benzothienyl, carbocyclyl, for example cyclopentyl and cyclohexyl, heterocyclyl, for example N-heterocyclyl, phenyl-(C₁₋₂ alkyl)-, for example benzyl, heteroaryl-(C₁₋₂ alkyl)- or —NR′R″ wherein R′ and R″ are as defined above.

More preferably, R⁵ is C₁₋₄ alkyl, C₁₋₄ alkoxy, phenyl, naphthyl, dihydrobenzofuranyl, benzodioxinyl, 9H-fluoren-9-onyl, indolyl, thienyl, furanyl, oxazolyl, isoxazolyl, pyrazolyl, pyridyl, benzothienyl, benzofuranyl, cyclopentyl, cyclohexyl, piperazinyl, piperidinyl, morpholinyl, phenyl-(C₁₋₂ alkyl)-, phenyl-CH₂—CH(OH)—, phenyl-CH(OH)—CH₂—, phenyl-(C₁₋₂ alkyl)-O—, 1H-benzo[d]imidazol-2(3H)-onyl or —NR′R″ wherein R′ and R″ are as defined above. Example of most preferred R⁵ groups are those wherein R⁵ is C₁₋₄ alkyl, phenyl, thienyl, furanyl, pyridyl, cyclopentyl, cyclohexyl, benzothienyl, dihydrobenzofuranyl, isoxazolyl, piperidinyl, for example N-piperidinyl, morpholinyl, for example N-morpholinyl, piperazinyl, for example N-piperazinyl, or —NR′R″ wherein R′ and R″ are as defined above.

Compounds produced by the preferred process of the present invention include those in which:

R¹ is C₁₋₆ alkyl or aryl;

R³ is halogen, hydroxy, C₁₋₄ alkyl, C₁₋₄ alkoxy, C₁₋₄ alkylthio, C₁₋₄ haloalkyl, C₁₋₄ haloalkoxy, amino, mono(C₁₋₄ alkyl)amino or di(C₁₋₄ alkyl)amino or, preferably, R³ is fluorine, chlorine, bromine, C₁₋₂ alkyl, C₁₋₂ alkoxy, C₁₋₂ alkylthio, C₁₋₂ haloalkyl, C₁₋₂ haloalkoxy, amino, mono(C₁₋₂ alkyl)amino or di(C₁₋₂ alkyl)amino;

n is 0, 1 or 2;

X is —NH—, —N(C₁₋₆ alkyl)-;

R⁴ is C₁₋₆ alkyl, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)-, aryl-C(O)—C(O)—, heteroaryl-C(O)—C(O)— or -ZR⁵;

Z is —CO—, —S(O)— or —S(O)₂—; and

R⁵ is C₁₋₆ alkyl, hydroxy, C₁₋₆ alkoxy, C₁₋₆ alkylthio, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)-, aryl-(C₁₋₄ hydroxyalkyl)-, heteroaryl-(C₁₋₄ hydroxyalkyl)-, carbocyclyl-(C₁₋₄ hydroxyalkyl)-, heterocyclyl-(C₁₋₄ hydroxyalkyl)-, aryl-(C₁₋₄ alkyl)-O—, heteroaryl-(C₁₋₄ alkyl)-O—, carbocyclyl-(C₁₋₄ alkyl)-O—, heterocyclyl-(C₁₋₄ alkyl)-O— or —NR′R″, wherein each R′ and R″ is the same or different and represents hydrogen, C₁₋₄ alkyl, aryl, heteroaryl, carbocyclyl, aryl-(C₁₋₄ alkyl)- or heteroaryl-(C₁₋₄ alkyl)-,

the aryl moiety in the R¹ group being unsubstituted or substituted by 1, 2 or 3 substituents selected from halogen, C₁₋₆ alkyl, C₁₋₆ alkoxy, C₁₋₆ alkylthio, C₁₋₆ haloalkyl or C₁₋₆ haloalkoxy;

the aryl and heteroaryl moieties in the groups R⁴ and R⁵ being unsubstituted or substituted by 1, 2 or 3 substituents selected from halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbomyl, amino, mono(C₁₋₆ alkyl)amino, di(C₁₋₆ alkyl)amino, —CO₂R′, —CONR′R″, —S(O)R′, —S(O)₂R′, —S(O)NR′R′, —S(O)₂NR′R″, —NH—S(O)₂R′ or —NH—CO—R′, wherein each R′ and R″ is the same or different and represents hydrogen or C₁₋₆ alkyl;

the carbocyclyl and heterocyclyl moieties in the groups R⁴ and R⁵ being unsubstituted or substituted by 1, 2 or 3 substituents selected from halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbomyl, amino, mono(C₁₋₆ alkyl)amino, di(C₁₋₆ alkyl)amino, oxo, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl; and

the alkyl moieties in the aryl-(C₁₋₄ alkyl)-, heteroaryl-(C₁₋₄ alkyl)-, carbocyclyl-(C₁₋₄ alkyl)-, heterocyclyl-(C₁₋₄ alkyl)- groups of R⁵ being unsubstituted or substituted by one or two hydroxy substituents.

Preferably, in these compounds produced by the preferred process of the present invention, the aryl, heteroaryl and carbocyclyl moieties in the groups R′ and R″ are unsubstituted or substituted by 1, 2 or 3 substituents selected from halogen, C₁₋₆ alkyl, C₁₋₆ alkoxy, C₁₋₆ alkylthio, C₁₋₆ haloalkyl, C₁₋₆ haloalkoxy, nitro and cyano.

Examples of compounds produced by the preferred process of the present invention are those wherein R¹, R³, X and n are as defined for the compounds produced by the preferred process of the present invention,

R⁴ is C₁₋₆ alkyl, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)- or -ZR⁵;

Z is —CO—, —S(O)— or —S(O)₂—; and

R⁵ is C₁₋₆ alkyl, hydroxy, C₁₋₆ alkoxy, C₁₋₆ alkylthio, aryl, heteroaryl, carbocyclyl, heterocyclyl, aryl-(C₁₋₄ alkyl)-, heteroaryl-(C₁₋₄ alkyl)-, carbocyclyl-(C₁₋₄ alkyl)-, heterocyclyl-(C₁₋₄ alkyl)- or —NR′R″, wherein each R′ and R″ is the same or different and represents hydrogen, C₁₋₄ alkyl, aryl, heteroaryl, carbocyclyl, aryl-(C₁₋₄ alkyl)- or heteroaryl-(C₁₋₄ alkyl)-,

the aryl, heteroaryl, carbocyclyl and heterocyclyl moieties in the groups R⁴ and R⁵ being unsubstituted or substituted by 1, 2 or 3 substituents selected from halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano.

Compounds produced by the preferred process of the present invention include those in which:

R¹ is C₁₋₆ alkyl or aryl;

R³ is halogen, hydroxy, C₁₋₄ alkyl, C₁₋₄ alkoxy, C₁₋₄ alkylthio, C₁₋₄ haloalkyl, C₁₋₄ haloalkoxy, amino, mono(C₁₋₄ alkyl)amino or di(C₁₋₄ alkyl)amino or, preferably, R³ is fluorine, chlorine, bromine, C₁₋₂ alkyl, C₁₋₂ alkoxy, C₁₋₂ alkylthio, C₁₋₂ haloalkyl, C₁₋₂ haloalkoxy,

n is 0, 1 or 2;

X is —NH—; and

R⁴ is —CO—R⁴′ or —CO—NH—R⁴′, wherein R^{4′}is a 5- or 6-membered heterocyclyl or heteroaryl ring which is substituted by a C₁₋₆ hydroxyalkyl group or a —(C₁₋₄ alkyl)-X₁—(C₁₋₄ alkyl)-X₂—(C₁₋₄ alkyl) group, wherein X₁ and X₂ are as defined above, or R^{4′} represents -A₁-Y-A₂, wherein:

A₁ is an aryl or heteroaryl group;

Y is a direct bond, a C₁₋₂ alkylene group, —SO₂— or —O—; and

A₂ is an aryl, heteroaryl, heterocyclyl or carbocyclyl group,

the aryl moiety in the R¹ group being unsubstituted or substituted by 1, 2 or 3 substituents selected from halogen, C₁₋₆ alkyl, C₁₋₆ alkoxy, C₁₋₆ alkylthio, C₁₋₆ haloalkyl and C₁₋₆ haloalkoxy groups,

the A₁ moiety being unsubstituted or substituted by 1 or 2 substituents selected from halogen, cyano, nitro, C₁₋₄ alkyl, C₁₋₄ haloalkyl and C₁₋₄ alkoxy substituents; and

the A₂ moiety being unsubstituted or substituted by one or two substituents which are selected from C₁₋₄ alkyl and halogen substituents when A₂ is a heteroaryl or aryl group and which are selected from C₁₋₄ alkyl, halogen and oxo substituents when A₂ is a carbocyclic or heterocyclyl group.

Typically, in this embodiment X is —CO—, —CO—NR′ or —S(O)₂—, wherein R′ is hydrogen or a C₁₋₂ alkyl group; and

R⁵ is a 5- or 6-membered heterocyclyl or heteroaryl ring which is substituted by a C₁-₆ hydroxyalkyl group or a —(C₁-₄ alkyl)-X₁—(C₁-₄ alkyl)-X₂—(C₁-₄ alkyl) group, wherein X₁ and X₂ are as defined above, or R⁵ represents -A₁-Y-A₂.

Compounds produced by the further preferred process of the present invention include those wherein:

R¹ is C₁₋₂ alkyl or phenyl;

R³ is methyl, trifluoromethyl, fluorine, chlorine or bromine;

n is 0 or 1;

X is —NH—;

R⁴ is C₁₋₄ alkyl, aryl, for example phenyl and dihydrobenzofuranyl, heteroaryl, for example thienyl, furanyl, isoxazolyl, pyridyl and benzothienyl, carbocyclyl, for example cyclopentyl and cyclohexyl, heterocyclyl, for example piperidinyl, morpholinyl and piperazinyl, phenyl-(C₁₋₂ alkyl)-, for example benzyl, heteroaryl-(C₁₋₂ alkyl)-, phenyl-C(O)—C(O)—, heteroaryl-C(O)—C(O)— or -ZR⁵, provided that when R⁴ is heterocyclyl it is attached via a carbon atom;

Z is —CO—, —S(O)— or —S(O)₂—; and

R⁵ is C₁₋₆ alkyl, C₁₋₆ alkoxy, C₁₋₆ alkylthio, aryl, for example phenyl, naphthyl, dihydrobenzofuranyl, benzodioxinyl, 9H-fluoren-9-onyl and indolyl, heteroaryl, for example thienyl, furanyl, oxazolyl, isoxazolyl, pyrazolyl, pyridyl, benzothienyl and benzofuranyl, carbocyclyl, for example cyclopentyl and cyclohexyl, heterocyclyl, for example piperazinyl, piperidinyl, morpholinyl and 1H-benzo[d]imidazol-2(3H)-onyl, phenyl-(C₁₋₂ alkyl)-, phenyl-(C₁₋₂ alkyl)-O—, phenyl-(C₁₋₂ hydroxyalkyl)-, heteroaryl-(C₁₋₂ hydroxyalkyl)-, heteroaryl-(C₁₋₂ alkyl)- or —NR′R″ wherein each R′ and R″ is the same or different and represents hydrogen, C₁₋₄ alkyl, phenyl, heteroaryl, for example thienyl, carbocyclyl, for example cyclohexyl or cyclopentyl, or phenyl-(C₁₋₄ alkyl)-,

the phenyl moiety in the R¹ group being unsubstituted or substituted by one or two substituents selected from fluorine, chlorine, bromine, C₁₋₄ alkyl, C₁₋₄ alkoxy, C₁₋₄ alkylthio, C₁₋₄ haloalkyl or C₁₋₄ haloalkoxy;

the aryl moieties in the groups R⁴ and R⁵ being unsubstituted or substituted by 1, 2 or 3 substituents selected from halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆ alkyl)amino, nitro, cyano, —CO₂R′, —S(O)R′, —S(O)₂R′ and —S(O)₂NR′R″, wherein each R′ and R″ is the same or different and represents hydrogen or C₁₋₄ alkyl;

the heteroaryl moieties in the groups R⁴ and R⁵ being unsubstituted or substituted by 1, 2 or 3 substituents selected from halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano; and

the carbocyclyl and heterocyclyl moieties in the groups R⁴ and R⁵ being unsubstituted or substituted by 1, 2 or 3 substituents selected from halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro, cyano and oxo; and

the alkyl moiety in the phenyl-(C₁₋₂ alkyl)- and heteroaryl-(C₁₋₂ alkyl)- groups of R⁵ being unsubstituted or substituted by a single hydroxy substituent.

Preferably, in these compounds produced by the further preferred process of the present invention, the phenyl, heteroaryl and carbocyclyl moieties in the groups R′ and R″ are unsubstituted or substituted by 1 or 2 substituents selected from fluorine, chlorine, bromine, C₁₋₄ alkyl, C₁₋₄ alkoxy, C₁₋₄ alkylthio, C₁₋₄ haloalkyl, C₁₋₄ haloalkoxy and nitro.

Examples of compounds produced by the further preferred process of the present invention include those wherein R¹, R³, X and n are as defined for the further preferred compounds of the invention,

R⁴ is C₁₋₄ alkyl, aryl, for example phenyl and dihydrobenzofuranyl, heteroaryl, for example thienyl, furanyl, isoxazolyl, pyridyl and benzothienyl, carbocyclyl, for example cyclopentyl and cyclohexyl, heterocyclyl, for example piperidinyl, morpholinyl and piperazinyl, phenyl-(C₁₋₂ alkyl)-, for example benzyl, heteroaryl-(C₁₋₂ alkyl)- or -ZR⁵, provided that when R⁴ is heterocyclyl it is attached via a carbon atom;

Z is —CO—, —S(O)— or —S(O)₂—; and

R⁵ is C₁₋₄ alkyl, aryl, for example phenyl and dihydrobenzofuranyl, heteroaryl, for example thienyl, furanyl, isoxazolyl, pyridyl and benzothienyl, carbocyclyl, for example cyclopentyl and cyclohexyl, heterocyclyl, for example N-heterocyclyl, phenyl-(C₁₋₂ alkyl)-, for example benzyl, heteroaryl-(C₁₋₂ alkyl)- or —NR′R″, wherein each R′ and R″ is the same or different and represents hydrogen, C₁₋₄ alkyl, cyclohexyl, cyclopentyl, phenyl or phenyl-CH₂—,

the aryl, heteroaryl, carbocyclyl and heterocyclyl moieties in the groups R⁵ and R⁶ being unsubstituted or substituted by 1 or 2 substituents selected from halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano.

As a further preference, in these compounds produced by the farther preferred process of the present invention, the cyclohexyl, cyclopentyl and phenyl moieties in the groups R′ and R″ are unsubstituted or substituted by 1 or 2 substituents selected from fluorine, chlorine, bromine, C₁₋₄ alkyl, C₁₋₄ alkoxy, C₁₋₄ is haloalkyl and nitro.

Compounds produced by the further preferred process of the present invention include those wherein:

R¹ is C₁₋₂ alkyl or phenyl;

R³ is methyl, trifluoromethyl, fluorine, chlorine or bromine;

n is 0 or 1;

X is —NH—; and

R⁴ is —CO—R⁴′ or —CO—NH—R⁴′, wherein R⁴′ is a 5- or 6-membered heterocyclyl or heteroaryl group which is substituted by a C₁-C₆ hydroxyalkyl group or a —(C₁-₄ alkyl)-NR′—(C₁-₄ alkyl)-SO₂—(C₁-₄ alkyl) group, wherein R′ is hydrogen or C₁-₂ alkyl, or R⁴ represents -A₁-Y-A₂, wherein:

A₁ is a phenyl group, a monocyclic 5- or 6-membered heteroaryl group or a 5- or 6-membered heteroaryl group fused to a monocyclic oxo-substituted 5- to 6-membered heterocyclyl group;

Y represents a direct bond, a C₁-C₂ alkylene moiety, —SO₂— or —O—; and

A₂ is a phenyl, 5- to 6-membered heteroaryl, 5- to 6-membered heterocyclyl or C₃-C₆ cycloalkyl group,

the phenyl moiety in the R¹ group being unsubstituted or substituted by one or two substituents selected from fluorine, chlorine, bromine, C₁₋₄ alkyl, C₁₋₄ alkoxy, C₁₋₄ alkylthio, C₁₋₄ haloalkyl or C₁₋₄ haloalkoxy;

the A₁ moiety being unsubstituted or substituted by 1 or 2 substituents selected from halogen, cyano, nitro, C₁-C₄ alkyl, C₁-C₄ haloalkyl and C₁-C₄ alkoxy substituents; and

the A₂ moiety being unsubstituted or substituted by 1 or 2 substituents which are selected from C₁-C₄ alkyl, halogen and oxo substituents when A₂ is a heterocyclyl or cycloalkyl group and which are selected from C₁-C₄ alkyl and halogen substituents when A₂ is a phenyl or heteroaryl group.

Typically, in this embodiment, X can also be —CO—, —CO—NR′— or —S(O)₂— wherein R′ is hydrogen or a C₁-C₂ alkyl group and R⁴ can be a 5- or 6-membered heterocyclyl or heteroaryl group which is substituted by a C₁-C₆ hydroxyalkyl group or a —(C₁₋₄ alkyl)-NR′—(C₁₋₄ alkyl)-SO₂—(C₁₋₄ alkyl) group, wherein R′ is hydrogen or C₁₋₂ alkyl, or R⁴ represents -A₁-Y-A₂.

The compounds produced by the particularly preferred process of the present invention include benzodiazepine derivatives of formula (I) as defined above, or pharmaceutically acceptable salts thereof, wherein:

R¹ is phenyl or methyl;

R³ is methyl or chlorine;

n is 0 or 1;

X is —NH—;

R⁴ is phenyl-CH₂—, furanyl-CH₂—, thienyl-C(O)—C(O)— or -ZR⁵;

Z is —CO— or —S(O)₂—; and

R⁵ is C₁₋₄ alkyl, C₁₋₄ alkoxy, phenyl, naphthyl, dihydrobenzofuranyl, benzodioxinyl, 9H-fluoren-9-onyl, indolyl, thienyl, furanyl, oxazolyl, isoxazolyl, pyrazolyl, pyridyl, benzothienyl, benzofuranyl, cyclopentyl, cyclohexyl, piperazinyl, piperidinyl, morpholinyl, phenyl-(C₁₋₂ alkyl)-, phenyl-CH₂—CH(OH)—, phenyl-CH(OH)—CH₂—, phenyl-(C₁₋₂ alkyl)-O—, 1H-benzo[d]imidazol-2(3H)-onyl or —NR′R″ wherein each R′ and R″ is the same or different and represents hydrogen, C₁₋₄ alkyl, phenyl, thienyl, cyclohexyl, cyclopentyl or phenyl-(CH₂)—,

the phenyl moiety in the group R¹ being unsubstituted or substituted by a single fluorine, chlorine, C₁₋₂ alkyl, C₁₋₂ alkoxy, C₁₋₂ alkylthio, C₁₋₂ haloalkyl or C₁₋₂ haloalkoxy substituent;

the aryl moieties in the groups R⁴ and R⁵ being unsubstituted or substituted by 1,2 or 3 substituents selected from fluorine, chlorine, bromine, iodine, C₁₋₄ alkyl, C₂₋₄ acyl, hydroxy, C₁₋₄ alkoxy, C₁₋₄ alkylthio, C₁₋₄ haloalkyl, C₁₋₄ haloalkoxy, amino, mono(C₁₋₄ alkyl)amino, di(C₁₋₄ alkyl)amino, nitro, —CO₂R′, —S(O)₂R′ and —S(O)₂NH₂, wherein R′ represents C₁₋₂ alkyl;

the heteroaryl moieties in the groups R⁴ and R⁵ being unsubstituted or substituted by 1 or 2 substituents selected from fluorine, chlorine, bromine, C₁₋₂ alkyl, C₁₋₂ haloalkyl and di(C₁₋₂ alkyl)amino; and

the heterocyclyl and carbocyclyl moieties in the R⁵ group being unsubstituted or substituted by 1 or 2 substituents selected from fluorine, chlorine, bromine, C₁₋₄ alkyl, C₁₋₄ alkoxy, C₁₋₄ haloalkyl and nitro.

Examples of compounds produced by the particularly preferred process of the present invention include benzodiazepine derivatives of formula (I) as defined above or pharmaceutically acceptable salts thereof, wherein:

R¹ is phenyl or methyl;

R³ is chlorine;

n is 0 or 1;

R⁴ is phenyl-CH₂—, furanyl-CH₂— or -ZR⁵;

Z is —CO— or —S(O)₂—; and

R⁵ is C₁₋₄ alkyl, phenyl, thienyl, furanyl, pyridyl, cyclopentyl, cyclohexyl, benzothienyl, dihydrobenzofuranyl, isoxazolyl, piperidinyl, for example N-piperidinyl, morpholinyl, for example N-morpholinyl, piperazinyl, for example N-piperazinyl, or —NR′R″, wherein each R′ and R″ is the same or different and represents hydrogen, C₁₋₄ alkyl, cyclohexyl, cyclopentyl, phenyl or phenyl-CH₂—, the phenyl, thienyl, furanyl, pyridyl, cyclopentyl, cyclohexyl, benzothienyl, dihydrobenzofuranyl, isoxazolyl, piperidinyl, morpholinyl and piperazinyl moieties in the groups R⁴ and R⁵ being unsubstituted or substituted by 1 or 2 substituents selected from fluorine, chlorine, bromine, C₁₋₄ alkyl, C₁₋₄ alkoxy, C₁₋₄ haloalkyl and nitro.

Preferably, in these compounds produced by the particularly preferred process of the present invention, the cyclohexyl, cyclopentyl and phenyl moieties of the groups R′ and R″ are unsubstituted or substituted by a single fluoro, chloro, methyl, methoxy or nitro substituent.

Compounds of the particularly preferred process of the present invention include benzodiazepine derivatives of formula (I) as defined above or pharmaceutically acceptable salts thereof, wherein:

X is —NH—; and

R⁴ is —CO—R⁴′ or —CO—NH—R⁴′, wherein R⁴′ is a 5- to 6-membered heteroaryl group, for example a furanyl group, which is substituted by —CH₂—OH or —(C₁₋₄ alkyl)-N(CH₃)—(C₁₋₄ alkyl)-SO₂—(C₁₋₄ alkyl) or R⁴ represents -A₁-Y-A₂, wherein:

A₁ is a phenyl, pyridyl, furanyl, thiazolyl, oxazolyl, isoxazolyl, thienyl or 1H-imidazo[4,5-b]pyridin-2-(3H)-one moiety, which is unsubstituted or substituted by 1 or 2 substituents selected from halogen, cyano, C₁-₂ alkyl, C₁-₂ haloalkyl and C₁-₂ alkoxy substituents;

Y is a direct bond, a C₁-₂ alkylene group, —SO₂— or —O—; and

A₂ is a piperazinyl, pyridyl, morpholinyl, pyrrolidinyl, piperidinyl, pyrazinyl, cydopropyl, phenyl or S,S-dioxo-thiomorpholino group, which is unsubstituted or substituted by a C₁₋₂ alkyl group.

Benzodiazepines which can be prepared by the process of the present invention are disclosed in UK patent application nos 0406280.8 and 0406282.4, from which the present application claims priority. These applications are incorporated herein by reference.

A benzodiazepine derivative of formula (I) produced by the process of the present invention may be converted into another such derivative by conventional means. In particular, one group [symbol] XR⁴ may be converted to another group [symbol] XR⁴. Interconversion may be carried out between benzodiazepine derivatives of formula (IIa), i.e. before the resolution step (a) of the process is carried out; between benzodiazepine derivatives of formula (II), i.e. after step (a) of the process but before the deprotection step (b); or between deprotected benzodiazepine derivatives of formula (I), i.e. after step (b) of the process.

In one embodiment of the process of the invention as defined above, wherein moiety [symbol] XR⁴ in formula (II) is sensitive to the conditions of deprotection of step (b), the process further comprises, prior to the deprotection step (b), converting the said moiety [symbol] XR⁴ into another moiety of formula [symbol] XR⁴ which is not sensitive to the conditions of deprotection.

In another embodiment of the process of the invention as defined above, the process further comprises:
(c) converting the moiety [symbol] XR⁴ in the benzodiazepine derivative of formula (I), which moiety is not sensitive to the conditions of deprotection used in the preceding step (b), into another moiety [symbol] XR⁴ which is either insensitive or sensitive to the conditions of deprotection used in step (b).

In a yet further embodiment of the process of the invention, in step (c), [symbol] XR⁴ is an amine (—NH₂) which is converted to a 2-fluorophenylurea (—NHC(O)NH-(2F-Ph)) group.

Examples of moieties [symbol] XR⁴ that maybe sensitive to deprotection conditions are —C(O)NH(C₁-₄ alkyl), —C(O)NH-aryl, —C(O)NH-heteraryl, —C(O)—(C₁-₄ alkyl), —C(O)-aryl and —C(O)-heteroaryl.

Interconversion of the group [symbol] XR⁴ is carried out by using suitable reagents and conditions. An example of a group [symbol] XR⁴ suitable for interconversion to another functional group [symbol] XR⁴ is an amine, i.e. X is —NH— and R⁴ is H. For example an amine may be transformed into a desired derivative, such as an amide or urea. Such an amide formation may be carried out using a suitable carboxylic acid and a coupling reagent, or a carbonyl chloride or other suitable reagent. Such a urea may be prepared using either a suitable isocyanate, or alternatively reaction with phosgene followed by a suitable amine. Suitable solvents for interconversion process are polar aprotic solvents, such as dichloromethane. Suitable coupling reagents are O-benzetriazol-1-yl-N,N,N′,N′-tetramethyluronium hexafluorophosphate (HBTU), N,N,N′,N′-tetramethyl-O-(benzotriazol-1-yl)uronium tetrafluoroborate (TBTU), or a 1-hydroxybenzotriazole (HOBT)/1-ethyl-3-(3-dimethylaminopropyl)carbodiimide hydrochloride (EDAC or EDCI) mixture, in the presence of a base, such as triethylamine.

A typical interconversion of the group [symbol] XR⁴ is from an amine (—NH₂) to 2-fluorophenylurea (—NHC(O)NH-(2F-Ph)). This may be effected by reaction of the amine compound with 2-fluorophenylisocyanate in the presence of triethylamine, in dichloromethane. Preferably such interconversion is effected before deprotection of the group R², i.e. between benzodiazepine derivatives of formula (II).

The process of the present invention typically further comprises interconverting a benzodiazepine derivative of formula (II) as defined above wherein the group [symbol] XR⁴ is sensitive to the reaction conditions of step (b), to yield another benzodiazepine derivative of formula (II) as defined above wherein the group [symbol] XR⁴ is not sensitive to the reaction conditions of step (b).

The interconversion of step (c) above may not be direct. For instance it may comprise interconverting a benzodiazepine derivative of formula (II) as defined above wherein the group [symbol] XR⁴ is not sensitive to the reaction conditions of deprotection step (b), to yield an intermediate benzodiazepine derivative of formula (II) as defined above; and subsequently interconverting that intermediate to yield another benzodiazepine derivative of formula (II) as defined above.

Benzodiazepine derivatives wherein X is —NH—, are particularly suited to this reaction strategy. This is because of the ease of conversion between amines, amides and ureas and the relative robustness of the amide group under common deprotection conditions for the moiety R². An example of a protecting group, R², for use in such a reaction is p-methoxybenzyl. Suitable groups —XR⁴ which are not sensitive to deprotection conditions are —NHC(O)—(C₁₋₆ alkyl), for example —NHC(O)—(C₁₋₄ alkyl), or more specifically —NHC(O)CH₃.

The above situation is illustrated by the following reaction scheme: 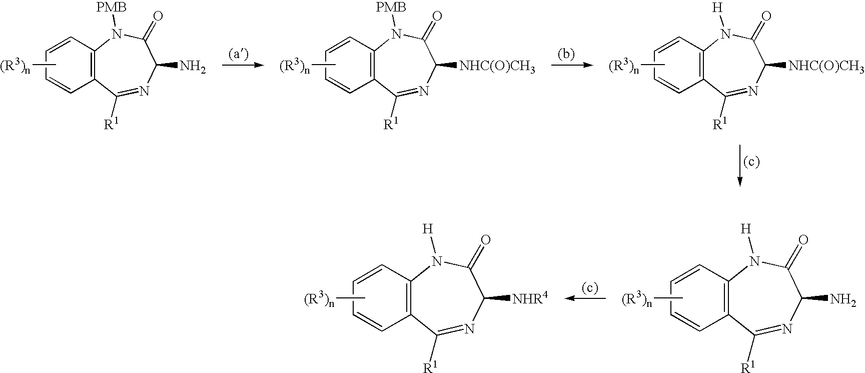
wherein PMB is paramethoxybenzyl.

Typically, the process of the invention involves the preparation of a compound of formula (II) in which R₄ is hydrogen, by (i) subjecting a corresponding racemic benzodiazepine of formula (IIa) to crystallisation induced dynamic resolution, (ii) deprotecting the compound of formula (II) to form a compound of formula (I), and then (iii) transforming the deprotected optically active benzodiazepine thereby obtained into another compound of formula (I) in which R₄ is other than hydrogen. Typically, in this embodiment, X is —NH—. Typically, in step (iii), the 3-substituent is transformed into a group —NH—CO—R⁵, wherein R⁵ is as defined above. Preferably, R₅ is —NH-(2F-phenyl).

The racemic benzodiazepine derivative of formula (IIa) as defined above may be produced by a process which comprises reducing a compound of formula (III): 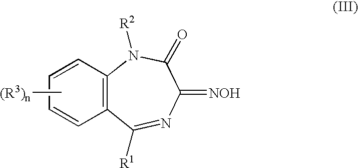
wherein R¹, R², R³ and n are as defined in claim 1, using hydrogen gas and a reducing catalyst in an inert solvent, to produce the desired compound of formula (IIa).

Typically the reaction is carried out at elevated temperature, for example from 30° C. to 100° C., preferably around 70° C. Typically the reaction is carried out at elevated pressure of hydrogen gas, for instance 40 psi to 200 psi, preferably around 130 psi. Typical solvents are alcohols, such as methanol and ethanol. A metal catalyst, such as a ruthenium catalyst is preferred.

The compound of formula (III) as defined above may be produced by a process which comprises treating a compound of formula (IV): 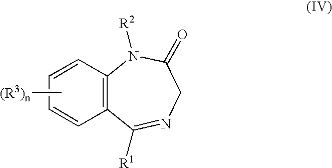
wherein R¹, R^{2,} R³, and n are as defined above, with isoamyl nitrite and a base in an inert solvent. Typical solvents are non-polar aromatic solvents, for example toluene. Strong bases are preferred, for instance sodium or potassium alkoxides, such as potassium tert-butoxide.

The compound of formula (IV) as defined above may be produced by a process which comprises submitting a compound of formula (V): 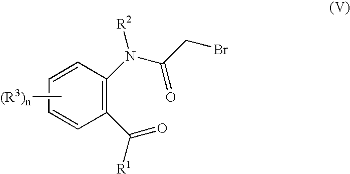
wherein R¹, R², R³ are as defined above, to cyclisation by treatment with ammonia to produce the desired compound of formula (IV).

The cyclisation is typically carried out by adding ammonia gas to an organic solvent such as an alcohol, for instance methanol, ethanol or isopropanol, and adding thereto the compound of formula (V). The reaction is typically carried out at a temperature of between 0° C. and room temperature, preferably around 15° C. to 18° C., followed by heating, for instance at the reflux temperature of the solvent.

The compound of formula (V) as defined above may be produced by treating a 2-aminophenone of formula (VI): 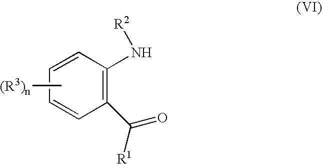
wherein R¹, R², R³ and n are as defined above, with bromoacetyl bromide in a suitable solvent. Typical solvents include polar aprotic solvents, such as dichloromethane. The reaction is typically carried out at a temperature of between −10° C. and room temperature, preferably around 0° C.

Typically in the compound of formula (V), R¹ is phenyl and n is 0. Typically in the compound of formula (VI), R¹ is phenyl and n is 0.

One embodiment of the process of the present invention is depicted by the reaction scheme below. 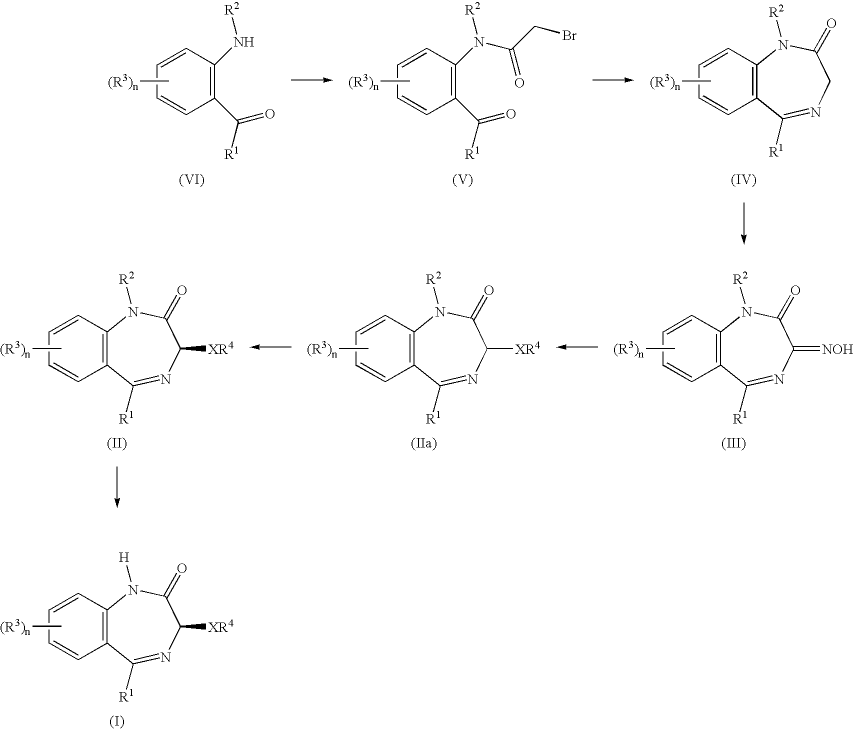
wherein the substituents R¹, R², R³, R⁵ and n are as defined above, the moiety —XR⁴ is —NH₂, and each reaction step is as defined above. The product compound is a benzodiazepine derivative of formula (I) or a pharmaceutically acceptable salt thereof. Typically n is 1 or 0, preferably 0. Typically R¹ is aryl, preferably phenyl. Typically R³ is halogen, preferably fluorine or chlorine.

A more preferred embodiment of the process of present invention is that depicted above wherein n is 0, R¹ is phenyl, X is —NH—, R² is 2-methoxybenzyl and R⁴ is —C(O)NH-(2-fluorophenyl).

A benzodiazepine derivative of formula (I) may be converted into a pharmaceutically acceptable salt, and a salt may be converted into a free compound by conventional methods. A pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutical acceptable bases include alkali metal (e.g. sodium or potassium) and alkaline earth metal (e.g. calcium or magnesium) hydroxides and organic bases such as alkyl amines, aralkyl amines or heterocyclic amines.

Examples of benzodiazepine derivative of formula (I) that can be produced by the process of the present invention include the R enantiomers and S enantiomers of:
(a) the following compounds:
N-(2-Oxo-5-phenyl-2,3-dihydro-1H-benzo[e][1,4]diazepin-3-yl)-acetamide;
1,1-Diethyl-3-(2-oxo-5-phenyl-2,3-dihydro-1H-benzo[e][1,4]diazepin-3-yl)-urea;
N-(2-Oxo-5-phenyl-2,3-dihydro-1H-benzo[e][1,4]diazepin-3-yl)-propionamide;
N-(2-Oxo-5-phenyl-2,3-dihydro-1H-benzo[e][1,4]diazepin-3-yl)-butyramide;
N-(2-Oxo-5-phenyl-2,3-dihydro-1H-benzo[e][1,4]diazepin-3-yl)-isobutyramide;
2,2-Dimethyl-N-(2-Oxo-5-phenyl-2,3-dihydro-1H-benzo[e][1,4]diazepin-3-yl)-propionamide;
Cyclopentanecarboxylic acid (2-oxo-5-phenyl-2,3-dihydro-1H-benzo[e][1,4]diazepin-3-yl)-amide;
Cyclohexanecarboxylic acid 2-oxo-5-phenyl-2,3-dihydro-1H-benzo[e][1,4]diazepin-3-yl)-amide;
3-Methoxy N-(2-oxo-5-phenyl-2,3-dihydro-1H-benzo[e][1,4]diazepin-3-yl)-benzamide;
4-Methoxy N-(2-oxo-5-phenyl-2,3-dihydro-1H-benzo[e][1,4]diazepin-3-yl)-benzamide;
2-Methoxy N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)-3-trifluoromethyl-benzamide;
N-(2-Oxo-5-phenyl-2,3-dihydro-1H-benzo[e][1,4]diazepin-3-yl)-benzamide;
Thiophene-2-carboxylic acid (2-oxo-5-phenyl-2,3-dihydro-1H-benzo[e][1,4]diazepin-3-yl)-3-amide;
Furan-2-carboxylic acid (2-oxo-5-phenyl-2,3-dihydro-1H-benzo[e][1,4]diazepin-3-yl)-amide;
Piperidine-1-carboxylic acid (2-oxo-5-phenyl-2,3-dihydro-1H-benzo[e][1,4]diazepin-3-yl)-amide;
Morpholine-4-carboxylic acid (2-oxo-5-phenyl-2,3-dihydro-1H-benzo[e][1,4]diazepin-3-yl)-amide;
4-Nitro-N-(2-oxo-5-phenyl-2,3-dihydro-1H-benzo[e][1,4]diazepin-3-yl)-benzamide;
3-Nitro-N-(2-oxo-5-phenyl-2,3-dihydro-1H-benzo[e][1,4]diazepin-3-yl)-benzamide;
4-Methyl-piperazine-1-carboxylic acid -(2-oxo-5-phenyl-2,3-dihydro-1H-benzo[e][1,4]diazepin-3-yl)-amide;
3,4-Dichloro-N-(2-oxo-5-phenyl-2,3-dihydro-1H-benzo[e][1,4]diazepin-3-yl)-benzamide;
N-(2-oxo-5-phenyl-2,3-dihydro-1H-benzo[e][1,4]diazepin-3-yl)-2-trifluoromethyl-benzamide;
4-Bromo-N-(2-oxo-5-phenyl-2,3-dihydro-1H-benzo[e][1,4]diazepin-3-yl)-benzamide;
2-Methyl-N-(2-oxo-5-phenyl-2,3-dihydro-1H-benzo[e][1,4]diazepin-3-yl)-benzamide;
2-Chloro-N-(2-oxo-5-phenyl-2,3-dihydro-1H-benzo[e][1,4]diazepin-3-yl)-benzamide;
2-Nitro-N-(2-oxo-5-phenyl-2,3-dihydro-1H-benzo[e][1,4]diazepin-3-yl)-benzamide;
2-Methoxy-4-nitro-N-(2-oxo-5-phenyl-2,3-dihydro-1H-benzo[e][1,4]diazepin-3-yl)-benzamide;
2-Methoxy-4-nitro-N-(2-oxo-5-phenyl-2,3-dihydro-1H-benzo[e][1,4]diazepin-3-yl)-benzamide
Benzo[b]thiophene-3-carboxylic acid (2-oxo-5-phenyl-2,3-dihydro-1H-benzo[e][1,4]diazepin-3-yl)-amide;
2,3-Dihydro-benzofuran-5-carboxylic acid (2-oxo-5-phenyl-2,3-dihydro-1H-benzo[e][1,4]diazepin-3-yl)-amide;
Isoxazole-5-carboxylic acid (2-oxo-5-phenyl-2,3-dihydro-1H-benzo[e][1,4]diazepin-3-yl)-amide;
Benzo[b]thiophene-2-carboxylic acid (2-oxo-5-phenyl-2,3-dihydro-1H-benzo[e][1,4]diazepin-3-yl)-amide;
Thiophen-3-carboxylic acid (2-oxo-5-phenyl-2,3-dihydro-1H-benzo[e][1,4]diazepin-3-yl)-amide;
N-(2-Oxo-5-phenyl-2,3-dihydro-1H-benzo[e][1,4]diazepin-3-yl)-isonicotinamide;
N-(2-Oxo-5-phenyl-2,3-dihydro-1H-benzo[e][1,4]diazepin-3-yl)-nicotinamide;
N-(2-Oxo-5-phenyl-2,3-dihydro-1H-benzo[e][1,4]diazepin-3-yl)-methanesulfonamide;
Propane-1-sulfonic acid-(2-oxo-5-phenyl-2,3-dihydro-1H-benzo[e][1,4]diazepin-3-yl)-amide;
Butane-1-sulfonic acid-(2-oxo-5-phenyl-2,3-dihydro-1H-benzo[e][1,4]diazepin-3-yl)-amide;
2-Bromo-N-(2-oxo-5-phenyl-2,3-dihydro-1H-benzo[e][1,4]diazepin-3-yl)-benzenesulfonamide;
3-Bromo-N-(2-oxo-5-phenyl-2,3-dihydro-1H-benzo[e][1,4]diazepin-3-yl)-benzenesulfonamide;
4-Bromo-N-(2-oxo-5-phenyl-2,3-dihydro-1H-benzo[e][1,4]diazepin-3-yl)-benzenesulfonamide;
2-Fluoro-N-(2-oxo-5-phenyl-2,3-dihydro-1H-benzo[e][1,4]diazepin-3-yl)-benzenesulfonamide;
3-(2-Nitro-benzylamino)-5-phenyl-1,3-dihydro-benzo[e][1,4]diazepin-2-one;
3-(3-Nitro-benzylamino)-5-phenyl-1,3-dihydro-benzo[e][1,4]diazepin-2-one;
3-(4-Nitro-benzylamino)-5-phenyl-1,3-dihydro-benzo[e][1,4]diazepin-2-one;
3-(2-Methoxy-benzylamino)-5-phenyl-1,3-dihydro-benzo[e][1,4]diazepin-2-one;
3-(3-Methoxy-benzylamino)-5-phenyl-1,3-dihydro-benzo[e][1,4]diazepin-2-one;
5-Phenyl-3-(2-trifluoromethyl-benzylamino)-1,3-dihydro-benzo[e][1,4]diazepin-2-one;
5-Phenyl-3-(3-trifluoromethyl-benzylamino)-1,3-dihydro-benzo[e][1,4]diazepin-2-one;
5-Phenyl-3-(4-trifluoromethyl-benzylamino)-1,3-dihydro-benzo[e][1,4]diazepin-2-one;
3-[(Furan-2-ylmethyl)-amino]-5-phenyl-1,3-dihydro-benzo[e][1,4]diazepin-2-one;
N-(7-Chloro-2-oxo-5-phenyl-2,3-dihydro-1H-benzo[e][1,4]diazepin-3-yl)-acetamide;
N-(7-Chloro-2-oxo-5-phenyl-2,3-dihydro-1H-benzo[e][1,4]diazepin-3-yl)-isobutyramide;
N-(7-Chloro-2-oxo-5-phenyl-2,3-dihydro-1H-benzo[e][1,4]diazepin-3-yl)-methanesulfonamide;
Furan-2-carboxylic acid (7-chloro-2-oxo-5-phenyl-2,3-dihydro-1H-benzo[e][1,4]diazepin-3-yl)-amide;
Thiophene-2-carboxylic acid (7-chloro-2-oxo-5-phenyl-2,3-dihydro-1H-benzo[e][1,4]diazepin-3-yl)-amide;
Cyclohexanecarboxylic acid (7-Chloro-2-oxo-5-phenyl-2,3-dihydro-1H-benzo[e][1,4]diazepin-3-yl)-amide;
N-(7-Chloro-2-oxo-5-phenyl-2,3-dihydro-1H-benzo[e][1,4]diazepin-3-yl)-2-methoxy-benzamide;
N-(7-Chloro-2-oxo-5-phenyl-2,3-dihydro-1H-benzo[e][1,4]diazepin-3-yl)-4-methoxy-benzamide;
N-(7-Chloro-2-oxo-5-phenyl-2,3-dihydro-1H-benzo[e][1,4]diazepin-3-yl)-2-nitro-benzamide;
2-(2-Methoxy-phenyl)N-(2-oxo-5-phenyl-2,3-dihydro-1H-benzo[e][1,4]diazepin-3-yl)-acetamide;
2-(3-Methoxy-phenyl)N-(2-oxo-5-phenyl-2,3-dihydro-1H-benzo[e][1,4]diazepin-3-yl)-acetamide;
2-(4-Methoxy-phenyl)N-(2-oxo-5-phenyl-2,3-dihydro-1H-benzo[e][1,4]diazepin-3-yl)-acetamide;
2-(4-Nitro-phenyl)N-(2-oxo-5-phenyl-2,3-dihydro-1H-benzo[e][1,4]diazepin-3-yl)-acetamide;
2-(3-Nitro-phenyl)N-(2-oxo-5-phenyl-2,3-dihydro-1H-benzo[e][1,4]diazepin-3-yl)-acetamide;
N-(2-Oxo-5-phenyl-2,3-dihydro-1H-benzo[e][1,4]diazepin-3-yl)-2-(2-trifluoromethyl-phenyl)-acetamide;
N-(2-Oxo-5-phenyl-2,3-dihydro-1H-benzo[e][1,4]diazepin-3-yl)-2-(3-trifluoromethyl-phenyl)-acetamide;
N-(2-Oxo-5-phenyl-2,3-dihydro-1H-benzo[e][1,4]diazepin-3-yl)-2-(4-trifluoromethyl-phenyl)-acetamide;
1-(2-Methoxy-phenyl)-3-(2-oxo-5-phenyl-2,3-dihydro-1H-benzo[e][1,4]diazepin-3-yl)-urea;

1-(2-Nitro-phenyl)-3-(2-oxo-5-phenyl-2,3-dihydro-1H-benzo[e][1,4]diazepin-3-yl)-urea;
1-(2-Chloro-phenyl)-3-(2-oxo-5-phenyl-2,3-dihydro-1H-benzo[e][1,4]diazepin-3-yl)-urea;
1-(4-Chloro-phenyl)-3-(2-oxo-5-phenyl-2,3-dihydro-1H-benzo[e][1,4]diazepin-3-yl)-urea;
1-(2-oxo-5-phenyl-2,3-dihydro-1H-benzo[e][1,4]diazepin-3-yl)-3-p-tolyl-urea;
1-(2-Fluoro-phenyl)-3-(2-oxo-5-phenyl-2,3-dihydro-1H-benzo[e][1,4]diazepin-3-yl)-urea;
1-(4-Fluoro-phenyl)-3-(2-oxo-5-phenyl-2,3-dihydro-1H-benzo[e][1,4]diazepin-3-yl)-urea;
4-Methanesulfonyl-2-methoxy-N-(2-oxo-5-phenyl-2,3-dihydro-1H-benzo[e][1,4]diazepin-3-yl)-benzamide;
5-Acetyl-2-ethoxy-N-(2-oxo-5-phenyl-2,3-dihydro-1H-benzo[e][1,4]diazepin-3-yl)-benzamide;
6-Fluoro-4H-benzo[1,3]dioxine-8-carboxylic acid (2-oxo-5-phenyl-2,3-dihydro-1H-benzo[e][1,4]diazepin-3-yl)-amide;
2-Methoxy-N-(2-oxo-5-phenyl-2,3-dihydro-1H-benzo[e][1,4]diazepin-3-yl)-4-trifluoromethyl-benzamide;
2,4,5-Trifluoro-N-(2-oxo-5-phenyl-2,3-dihydro-1H-benzo[e][1,4]diazepin-3-yl)-benzamide;
2-Hydroxy-N-(2-oxo-5-phenyl-2,3-dihydro-1H-benzo[e][1,4]diazepin-3-yl)-benzamide;
1H-Indole-7-carboxylic acid (2-oxo-5-phenyl-2,3-dihydro-1H-benzo[e][1,4]diazepin-3-yl)-amide;
3-Methoxy-naphthalene-2-carboxylic acid (2-oxo-5-phenyl-2,3-dihydro-1H-benzo[e][1,4]diazepin-3-yl)-amide;
N-[7-Chloro-5-(2-fluoro-phenyl)-2-oxo-2,3-dihydro-1H-benzo[e][1,4]diazepine-3-yl]-4-methoxoy-benzamide;
1-(2-Fluoro-benzyl)-3-(2-oxo-5-phenyl-2,3-dihydro-1H-benzo[e][1,4]diazepin-3-yl)urea;
1-(4-Methoxy-benzyl)-3-(2-oxo-5-phenyl-2,3-dihydro-1H-benzo[e][1,4]diazepin-3-yl)-urea;
1-(3-Methyl-benzyl)-3-(2-oxo-5-phenyl-2,3-dihydro-1H-benzo[e][1,4]diazepin-3-yl)-urea;
1-(2-Oxo-5-phenyl-2,3-dihydro-1H-benzo[e][1,4]diazepin-3-yl)-3-(4-trifluoromethyl-phenyl)-urea;
4-Chloro-2-methoxy-N-(2-oxo-5-phenyl-2,3-dihydro-1H-benzo[e][1,4]diazepin-3-yl)-benzamide;
4-Methoxy-3-nitro-N-(2-oxo-5-phenyl-2,3-dihydro-1H-benzo[e][1,4]diazepin-3-yl)benzamide;
3-Methoxy-2-nitro-N-(2-oxo-5-phenyl-2,3-dihydro-1H-benzo[e][1,4]diazepin-3-yl)-benzamide;
5-Chloro-2-methoxy-N-(2-oxo-5-phenyl-2,3-dihydro-1H-benzo[e][1,4]diazepin-3-yl)benzamide;
5-Fluoro-2-methoxy-N-(2-oxo-5-phenyl-2,3-dihydro-1H-benzo[e][1,4]diazepin-3-yl)-benzamide;
2-Methoxy-4-nitro-N-(2-oxo-5-phenyl-2,3-dihydro-1H-benzo[e][1,4]diazepin-3-yl)-benzamide;
5-Methoxy-2-nitro-N-(2-oxo-5-phenyl-2,3-dihydro-1H-benzo [e][1,4]diazepin-3-yl)-benzamide;
3-Methoxy-4-nitro-N-(2-oxo-5-phenyl-2,3-dihydro-1H-benzo[e][1,4]diazepin-3-yl)-benzamide;
3-(2-Methoxy-phenyl)-N-(2-oxo-5-phenyl-2,3-dihydro-1H-benzo[e][1,4]diazepin-3-yl)propionamide;
3-(3-Methoxy-phenyl)-N-(2-oxo-5-phenyl-2,3-dihydro-1H-benzo[e][1,4]diazepin-3-yl)-propionamide;
3-(4-Methoxy-phenyl)-N-(2-oxo-5-phenyl-2,3-dihydro-1H-benzo[e][1,4]diazepin-3-yl)-propionamide;
N-[5-(3-Chloro-phenyl)-2-oxo-2,3-dihydro-1H-benzo[e][1,4]diazepin-3-yl]-2-methoxy-benzamide;
N-[5-(3-Chloro-phenyl)-2-oxo-5-phenyl-2,3-dihydro-1H-benzo[e][1,4]diazepin-3-yl]-4-methoxy-benzamide;
N-[5-(3-Chloro-phenyl)-2-oxo-5-phenyl-2,3-dihydro-1H-benzo[e][1,4]diazepin-3-yl]-2-nitro-benzamide;
N-[5-(3-Chloro-phenyl)-2-oxo-5-phenyl-2,3-dihydro-1H-benzo[e][1,4]diazepin-3-yl]-4-nitro-benzamide;
4-Methoxy-N-[2-oxo-5-(4-trifluoromethyl-phenyl)-2,3-dihydro-1H-benzo[e][1,4]diazepin-3-yl]-benzamide;
2-Methoxy-N-[2-oxo-5-(3-trifluoromethyl-phenyl)-2,3-dihydro-1H-benzo[e][1,4]diazepin-3-yl]-benzamide;
4-Methoxy-N-[2-oxo-5-(3-trifluoromethyl-phenyl)-2,3-dihydro-1H-benzo[e][1,4]diazepin-3-yl]-benzamide;
2-Ethoxy-N-(2-oxo-5-phenyl-2,3-dihydro-1H-benzo[e][1,4]diazepin-3-yl)-benzamide;
2,4-Dimethoxy-N-(2-oxo-5-phenyl-2,3-dihydro-1H-benzo[e][1,4]diazepin-3-yl)-benzamide;
2-Bromo-5-methoxy-N-(2-oxo-5-phenyl-2,3-dihydro-1H-benzo[e][1,4]diazepin-3-yl)-benzamide;
2-Methoxy-N-[5-(3-mehtoxy-phenyl)-2-oxo-2,3-dihydro-1H-benzo[e][1,4]diazepin-3-yl]-benzamide
N-[5-(3-Methoxy-phenyl)-2-oxo-5-phenyl-2,3-dihydro-1H-benzo[e][1,4]diazepin-3-yl]-4-nitro-benzamide;
2-Methoxy-N-(8-methyl-2-oxo-5-phenyl-2,3-dihydro-1H-benzo[e][1,4]diazepin-3-yl)-benzamide;
2-Chloro-4-methanesulfonyl-N-(2-oxo-5-phenyl-2,3-dihydro-1H-benzo[e][1,4]diazepin-3-yl)-benzamide;
2-Dimethylamino-N-(2-oxo-5-phenyl-2,3-dihydro-1H-benzo[e][1,4]diazepin-3-yl)-benzamide;
(2-Oxo-5-phenyl-2,3-dihydro-1H-benzo[e][1,4]diazepin-3-yl)-carbamic acid benzyl ester;
1-(3,5-Dimethyl-phenyl)-3-(2-oxo-5-phenyl-2,3-dihydro-1H-benzo[e][1,4]diazepin-3-yl)-urea;
1-(2-Oxo-5-phenyl-2,3-dihydro-1H-benzo[e][1,4]diazepin-3-yl)-3-(4-trifluoromethoxy-phenyl)-urea;
1-(4-Bromo-2-trifluoromethyl-phenyl)-3-(2-oxo-5-phenyl-2,3-dihydro-1H-benzo[e][1,4]diazepin-3-yl)-urea;
1-(4-Bromo-benzyl)-3-(2-oxo-5-phenyl-2,3-dihydro-1H-benzo[e][1,4]diazepin-3-yl)-urea;
1-(2,3-Dichloro-phenyl)-3-(2-oxo-5-phenyl-2,3-dihydro-1H-benzo[e][1,4]diazepin-3-yl)-urea;
1-(2,6-Dimethyl-phenyl)-3-(2-oxo-5-phenyl-2,3-dihydro-1H-benzo[e][1,4]diazepin-3-yl)-urea;
1-(2-Chloro-6-methyl-phenyl)-3-(2-oxo-5-phenyl-2,3-dihydro-1H-benzo[e][1,4]diazepin-3-yl)-urea;
1-(4-Nitro-phenyl)-3-(2-oxo-5-phenyl-2,3-dihydro-1H-benzo[e][1,4]diazepin-3-yl)-urea;
1-(2-Methylsulfanyl-phenyl)-3-(2-oxo-5-phenyl-2,3-dihydro-1H-benzo[e][1,4]diazepin-3-yl)-urea;
1-(2,6-Dichloro-phenyl)-3-(2-oxo-5-phenyl-2,3-dihydro-1H-benzo[e][1,4]diazepin-3-yl)-urea;
5-tert-Butyl-2-methoxy-N-(2-oxo-5-phenyl-2,3-dihydro-1H-benzo[e][1,4]diazepin-3-yl)-benzamide;
2,5-Dimethoxy-N-(2-oxo-5-phenyl-2,3-dihydro-1H-benzo [e][1,4]diazepin-3-yl)-benzamide;
1-(2,6-Difluoro-phenyl)-3-(2-oxo-5-phenyl-2,3-dihydro-1H-benzo[e][1,4]diazepin-3-yl)-urea;
1-(3-Fluoro-phenyl)-3-(2-oxo-5-phenyl-2,3-dihydro-1H-benzo[e][1,4]diazepin-3-yl)-urea;

1-(3-Methoxy-phenyl)-3-(2-oxo-5-phenyl-2,3-dihydro-1H-benzo[e][1,4]diazepin-3yl)-urea;
1-(2-Oxo-5-phenyl-2,3-dihydro-1H-benzo[e][1,4]diazepin-3-yl)-3-(3-trifluoromethyl-phenyl)-urea;
1-(3-Chloro-phenyl)-3-(2-oxo-5-phenyl-2,3-dihydro-1H-benzo[e][1,4]diazepin-3-yl)-urea;
2-Methoxy-4-methylsulfanyl-N-(2-oxo-5-phenyl-2,3-dihydro-1H-benzo[e][1,4]diazepin-3-yl)-benzamide;
4-Methanesulfonyl-N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)terephthalamic acid methyl ester;
2-Fluoro-N-(2-oxo-5-phenyl-2,3-dihydro-1H-benzo[e][1,4]diazepin-3-yl)-benzamide;
2,6-Difluoro-N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)-2-propoxy-benzamide;
2-Iodo-N-(2-oxo-5-phenyl-2,3-dihydro-1H-benzo[e][1,4]diazepin-3-yl)-benzamide;
3-Methoxy-N-(2-oxo-5-phenyl-2,3-dihydro-1H-benzo[e][1,4]diazepin-3-yl)-terephthalamic acid methyl ester;
4-Amino-5-chloro-2-methoxy-N-(2-oxo-5-phenyl-2,3-dihydro-1H-benzo[e][1,4]diazepin-3-yl)-benzamide;
1-(2-Oxo-5-phenyl-2,3-dihydro-1H-benzo[e][1,4]diazepin-3-yl)-3-m-tolyl-urea;
2-Methylsulfanyl-N-(2-oxo-5-phenyl-2,3-dihydro-1H-benzo[e][1,4]diazepin-3-yl)-benzamide;
2-Methoxy-N-(2-oxo-5-phenyl-2,3-dihydro-1H-benzo[e][1,4]diazepin-3-yl)-5-sulfamoyl-benzamide;
2-Hydroxy-N-(2-oxo-5-phenyl-2,3-dihydro-1H-benzo[e][1,4]diazepin-3-yl)-3-phenyl-propionamide
3-Hydroxy-N-(2-oxo-5-phenyl-2,3-dihydro-1H-benzo[e][1,4]diazepin-3-yl)-3-phenyl-propionamide;
3-(2-Fluoro-phenyl)-1-methyl-1-(2-oxo-5-phenyl-2,3-dihydro-1H-benzo[e][1,4]diazepin-3-yl)-urea;
2-Methoxy-N-methyl-4-nitro-N-(2-oxo-5-phenyl-2,3-dihydro-1H-benzo[e][1,4]diazepin-3-yl)-benzamide;
1-tert-Butyl-3-(2-oxo-5-phenyl-2,3-dihydro-1H-benzo[e][1,4]diazepin-3-yl)-urea;
1-Cycloheyl-3-(2-oxo-5-phenyl-2,3-dihydro-1H-benzo[e][1,4]diazepin-3-yl)-urea;
1-Ethyl-3-(2-oxo-5-phenyl-2,3-dihydro-1H-benzo [e][1,4]diazepin-3-yl)-urea;
1-Butyl-3-(2-oxo-5-phenyl-2,3-dihydro-1H-benzo[e][1,4]diazepin-3-yl)-urea;
4,5-Dimethyl-furan-2-carboxylic acid (2-oxo-5-phenyl-2,3-dihydro-1H-benzo[e][1,4]diazepin-3-yl)amide;
Piperidine-1-carboxylic acid (7-chloro-2-oxo-5-phenyl-2,3-dihydro-1H-benzo[e][1,4]diazepin-3-yl)-amide;
N-[5-(3-Chloro-phenyl)-2-oxo-5-phenyl-2,3-dihydro-1H-benzo[e][1,4]diazepin-3-yl)acetamide;
N-[5-(3-Chloro-phenyl)-2-oxo-5-phenyl-2,3-dihydro-1H-benzo[e][1,4]diazepin-3-yl]-isobutyramide;
Furan-2-carboxylic acid [5-(3-chloro-phenyl)-2-oxo-5-phenyl-2,3-dihydro-1H-benzo[e][1,4]diazepin-3-yl]-amide;
Thiophene-2-carboxylic acid [5-(3-chloro-phenyl)-2-oxo-5-phenyl-2,3-dihydro-1H-benzo[e][1,4]diazepin-3-yl]-amide;
Cyclohexanecarboxylic acid [5-(3chloro-phenyl)-2-oxo-5-phenyl-2,3-dihydro-1H-benzo[e][1,4]diazepin-3-yl]-amide;
Piperidine-1-carboxylic acid [5-(3-chloro-phenyl)-2-oxo-5-phenyl-2,3-dihydro-1H-benzo[e][1,4]diazepin-3-yl]-amide;
N-[5-(3-Chloro-phenyl)-2-oxo-5-phenyl-2,3-dihydro-1H-benzo[e][1,4]diazepin-3-yl]isonicotinamide;
5-Methyl-furan-2-carboxylic acid (2-oxo-5-phenyl-2,3-dihydro-1H-benzo[e][1,4]diazepin-3-yl)-amide;
Pyrazine-2-carboxylic acid (2-oxo-5-phenyl-2,3-dihydro-1H-benzo[e][1,4]diazepin-3-yl)-amide;
N-[5-(3-Methoxy-phenyl)-2-oxo-5-phenyl-2,3-dihydro-1H-benzo[e][1,4]diazepin-3-yl]-isobutyramide;
Thiophene-2-carboxylic acid [5-(3-methoxy-phenyl)-2-oxo-5-phenyl-2,3-dihydro-1H-benzo[e][1,4]diazepin-3-yl]-amide;
Cyclohexanecarboxylic acid [5-(3-methoxy-phenyl)-2-oxo-5-phenyl-2,3-dihydro-1H-benzo[e][1,4]diazepin-3-yl]-amide;
Piperidine-1-carboxylic acid [5-(3-methoxy-phenyl)-2-oxo-5-phenyl-2,3-dihydro-1H-benzo[e][1,4]diazepin-3-yl]-amide;
Piperidine-4-carboxylic acid [5-(3-methoxy-phenyl)-2-oxo-5-phenyl-2,3-dihydro-1H-benzo[e][1,4]diazepin-3-yl]-amide;
Cyclohexanecarboxylic acid (8-chloro-2-oxo-5-phenyl-2,3-dihydro-1H-benzo[e][1,4]diazepin-3-yl)-amide;
Thiophene-2-carboxylic acid (8-methyl-2-oxo-5-phenyl-2,3-dihydro-1H-benzo[e][1,4]diazepin-3-yl)-amide;
1-(2-Oxo-5-phenyl-2,3-dihydro-1H-benzo[e][1,4]diazepin-3-yl)-3-thiophene-2-yl-urea;
1-(2-Oxo-5-phenyl-2,3-dihydro-1H-benzo[e][1,4]diazepin-3-yl)-3-thiophene-3-yl-urea;
Pyridine-2-carboxylic acid (2-oxo-5-phenyl-2,3-dihydro-1H-benzo[e][1,4]diazepin-3-yl)-amide;
1H-Pyrazole-4-carboxylic acid (2-oxo-5-phenyl-2,3-dihydro-1H-benzo[e][1,4]diazepin-3-yl)-amide;
6-Dimethylamino-N-(2-oxo-5-phenyl-2,3-dihydro-1H-benzo[e][1,4]diazepin-3-yl)-nicotinamide;
2-Ethoxy-naphthalene-1-carboxylic acid (2-oxo-5-phenyl-2,3-dihydro-1H-benzo[e][1,4]diazepin-3-yl)-amide;
9-Oxo-9H-fluorene-1-carboxylic acid (2-oxo-5-phenyl-2,3-dihydro-1H-benzo[e][1,4]diazepin-3-yl)-amide;
2-Oxo-2,3-dihydro-benzoimidazole-1-carboxylic acid (2-oxo-5-phenyl-2,3-dihydro-1H-benzo[e][1,4]diazepin-3-yl)-amide;
(2-Oxo-5-phenyl-2,3-dihydro-1H-benzo[e][1,4]diazepin-3-yl)carbamic acid tert-butyl ester;
4,5-Dibromo-furan-2-carboxylic acid (2-oxo-5-phenyl-2,3-dihydro-1H-benzo[e][1,4]diazepin-3-yl)-amide;
Benzofuran-2-carboxylic acid (2-oxo-5-phenyl-2,3-dihydro-1H-benzo[e][1,4]diazepin-3-yl)-amide;
(2-Oxo-5-phenyl-2,3-dihydro-1H-benzo[e][1,4]diazepin-3-yl)-carbamic acid methyl ester;
(2-Oxo-5-phenyl-2,3-dihydro-1H-benzo[e][1,4]diazepin-3-yl)-carbamic acid ethyl ester;
(2-Oxo-5-phenyl-2,3-dihydro-1H-benzo[e][1,4]diazepin-3-yl)-carbamic acid isobutyl ester; and
2-Oxo-N-(2-oxo-5-phenyl-2,3-dihydro-1H-benzo[e][1,4]diazepin-3-yl)-2-thiophene-2-yl-acetamide; and
(b) one of the following compounds and the N-oxides thereof:
6-(4-Methyl-piperazin-1-yl)-N-(2-oxo-5-phenyl-2,3-dihydro-1H-benzo[e][1,4]diazepin-3-yl)-nicotinamide;
3,4,5,6-Tetrahydro-2H-[1,2′]bipyridinyl-5′-carboxylic acid (2-oxo-5-phenyl-2,3-dihydro-1H-benzo[e][1,4]diazepin-3-yl)-amide;
2-(1,1-Dioxo-1λ6-thiomorpholin-4-yl)-N-(2-oxo-5-phenyl-2,3-dihydro-1H-benzo[e][1,4]diazepin-3-yl-benzamide;
2-Chloro-4-morpholin-4-yl-N-(2-oxo-5-phenyl-2,3-dihydro-1H-benzo[e][1,4]diazepin-3-yl)-benzamide;
2-(1,1-Dioxo-1λ6-thiomorpholin-4-yl)-4-fluoro-(2-oxo-5-phenyl-2,3-dihydro-1H-benzo[e][1,4]diazepin-3-yl-benzamide;
5-Chloro-2-(1,1-dioxo-1λ6-thiomorpholin-4-yl)-N-(2-oxo-5-phenyl-2,3-dihydro-1H-benzo[e][1,4]diazepin-3-yl)-benzamide;
2-(1,1-Dioxo-1λ6-thiomorpholin-4-yl)-5-fluoro-N-(2-oxo-5-phenyl-2,3-dihydro-1H-benzo[e][1,4]diazepin-3-yl)-benzamide;
5-(4-Methyl-piperazin-1-ylmethyl)-furan-2-carboxylic acid (2-oxo-5-phenyl-2,3-dihydro-1H-benzo[e][1,4]diazepin-3-yl)-amide;
5-Pyrrolidin-1-ylmethyl-furan-2-carboxylic acid (2-oxo-5-phenyl-2,3-dihydro-1H-benzo[e][1,4]diazepin-3-yl)-amide;
5-Piperidin-1-ylmethyl-furan-2-carboxylic acid (2-oxo-5-phenyl-2,3-dihydro-1H-benzo[e][1,4]diazepin-3-yl)-amide;
5-Dimethylaminomethyl-furan-2-carboxylic acid (2-oxo-5-phenyl-2,3-dihydro-1H-benzo[e][1,4]diazepin-3-yl)-amide;
4-Fluoro-N-(2-oxo-5-phenyl-2,3-dihydro-1H-benzo[e][1,4]diazepin-3-yl)-2-piperidin-1-yl-benzamide;
4-Fluoro-2-morpholino-4-yl-N-(2-oxo-5-phenyl-2,3-dihydro-1H-benzo[e][1,4]diazepin-3-yl)-benzamide;
4-Cyano-N-(2-oxo-5-phenyl-2,3-dihydro-1H-benzo[e][1,4]diazepin-3-yl)-2-pyrrolidin-1-yl-benzamide;
4-Cyano-N-(2-oxo-5-phenyl-2,3-dihydro-1H-benzo[e][1,4]diazepin-3-yl)-piperidine-1-yl-benzamide;
N-(2-Oxo-5-phenyl-2,3-dihydro-1H-benzo[e][1,4]diazepin-3-yl)-2-pyrrolidin-1-yl-4-trifluoromethyl-benzamide;
N-(2-Oxo-5-phenyl-2,3-dihydro-1H-benzo[e][1,4]diazepin-3-yl)-2-piperidin-1-yl-4-trifluoromethyl-benzamide;
2-Morpholin-4-yl-N-(2-oxo-5-phenyl-2,3-dihydro-1H-benzo[e][1,4]diazepin-3-yl)-4-trifluoromethyl-benzamide;
N-(2-Oxo-5-phenyl-2,3-dihydro-1H-benzo[e][1,4]diazepin-3-yl)-2-pyrrolidin-1-yl-5-trifluoromethyl-benzamide;
2-Morpholin-4-yl-N-(2-oxo-5-phenyl-2,3-dihydro-1H-benzo[e][1,4]diazepin-3-yl)-5-trifluoromethyl-benzamide;
2-Morpholin-4-yl-N-(2-oxo-5-phenyl-2,3-dihydro-1H-benzo[e][1,4]diazepin-3-yl)-nicotinamide;
2-(1,1-Dioxo-1λ6-thiomorpholin-4-yl)-N-(2-oxo-5-phenyl-2,3-dihydro-1H-benzo[e][1,4]diazepin-3-yl)-nicotinamide;
2-(1,1-Dioxo-1λ6-thiomorpholin-4-yl)-3-methyl-N-(2-oxo-5-phenyl-2,3-dihydro-1H-benzo[e][1,4]diazepin-3-yl)-benzamide;
2-(l1,1-Dioxo-1λ6-thiomorpholin-4-yl)-4-methyl-N-(2-oxo-5-phenyl-2,3-dihydro-1H-benzo[e][1,4]diazepin-3-yl)-benzamide;
2-(1,1-Dioxo-1λ6-thiomorpholin-4-yl)-6-methyl-N-(2-oxo-5-phenyl-2,3-dihydro-1H-benzo[e][1,4]diazepin-3-yl)-benzamide;
2-Chloro-6-(1,1-dioxo-1λ6-thiomorpholin-4-yl)-N-(2-oxo-5-phenyl-2,3-dihydro-1H-benzo[e][1,4]diazepin-3-yl)-benzamide;
3-Cyclopropyl-2-oxo-2,3-dihydro-imidazo[4,5-b]pyridine-1-carboxylic acid (2-oxo-5-phenyl-2,3-dihydro-1H-benzo[e][1,4]diazepin-3-yl)-amide;
3-(4-Methyl-piperazine-1-sulfonyl)-N-(2-oxo-5-phenyl-2,3-dihydro-1H-benzo[e][1,4]diazepin-3-yl)-benzamide;
4-(4-Methyl-piperazin-1-yl)-N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)-3-(piperidine-1-sulfonyl)-benzamide;
3-(Morpholine-4-sulfonyl)-N-(2-oxo-5-phenyl-2,3-dihydro-1H-benzo[e][1,4]diazepin-3-yl)-benzamide;
5-Morpholin-4-ylmethyl-furan-2-carboxylic acid (2-oxo-5-phenyl-2,3-dihydro-1H-benzo[e][1,4]diazepin-3-yl)-amide;
5-Hydroxymethyl-furan-2-carboxylic acid (2-oxo-5-phenyl-2,3-dihydro-1H-benzo[e][1,4]diazepin-3-yl)-amide;
5-(1,1-Dioxo-1λ6-thiomorpholin-4-ylmethyl)-furan-2-carboxylic acid (2-oxo-5-phenyl-2,3-dihydro-1H-benzo[e][1,4]diazepin-3-yl)-amide;
2-Chloro-4-(1,1-dioxo-1λ6-thiomorpholin-4-yl)-N-(2-oxo-5-phenyl-2,3-dihydro-1H-benzo[e][1,4]diazepin-3-yl)-benzamide;
2-Chloro-5-(1,1-dioxo-1λ6-thiomorpholin-4-yl)-N-(2-oxo-5-phenyl-2,3-dihydro-1H-benzo[e][1,4]diazepin-3-yl)-benzamide;
5-{[(2-Methanesulfonyl-ethyl)-methyl-amino]-methyl}-furan-2-carboxylic acid (2-oxo-5-phenyl-2,3-dihydro-1H-benzo[e][1,4]diazepin-3-yl-amide;
2-Pyridin-3-yl-thiazole-4-carboxylic acid (2-oxo-5-phenyl-2,3-dihydro-1H-benzo[e][1,4]diazepin-3-yl)-amide;
2-Pyridin-4-yl-thiazole-4-carboxylic acid (2-oxo-5-phenyl-2,3-dihydro-1H-benzo[e][1,4]diazepin-3-yl)-amide;
4-Methyl-2-pyrazin-2-yl-thiazole-5-carboxylic acid (2-oxo-5-phenyl-2,3-dihydro-1H-benzo[e][1,4]diazepin-3-yl)-amide;
2-Morpholin-4-ylmethyl-furan-3-carboxylic acid (2-oxo-5-phenyl-2,3-dihydro-1H-benzo[e][1,4]diazepin-3-yl)-amide;
3-Morpholin-4-ylmethyl-N-(2-oxo-5-phenyl-2,3-dihydro-1H-benzo[e][1,4]diazepin-3-yl)-benzamide;
5-Morpholin-4-ylmethyl-isoxazole-3-carboxylic acid (2-oxo-5-phenyl-2,3-dihydro-1H-benzo[e][1,4]diazepin-3-yl)-amide;
3-Morpholin-4-ylmethyl-furan-2-carboxylic acid (2-oxo-5-phenyl-2,3-dihydro-1H-benzo[e][1,4]diazepin-3-yl)-amide;
5-Pyridin-2-yl-thiophene-2-carboxylic acid (2-oxo-5-phenyl-2,3-dihydro-1H-benzo[e][1,4]diazepin-3-yl)-amide;
2-Methyl-4-(morpholin-4-sulfonyl)-furan-3-carboxylic acid (2-oxo-5-phenyl-2,3-dihydro-1H-benzo[e][1,4]diazepin-3-yl)-amide;
6-Morpholin-4-yl-N-(2-oxo-5-phenyl-2,3-dihydro-1H-benzo[e][1,4]diazepin-3-yl)-nicotinamide;
3-Morpholin-4-ylmethyl-thiophene-2-carboxylic acid (2-oxo-5-phenyl-2,3-dihydro-1H-benzo[e][1,4]diazepin-3-yl)-amide;
5-Morpholin-4-ylmethyl-thiophene-2-carboxylic acid (2-oxo-5-phenyl-2,3-dihydro-1H-benzo[e][1,4]diazepin-3-yl)-amide;
2-Morpholin-4-yl-N-(2-oxo-5-phenyl-2,3-dihydro-1H-benzo[e][1,4]diazepin-3-yl)-benzamide;
5-Phenyl-oxazole-4-carboxylic acid (2-oxo-5-phenyl-2,3-dihydro-1H-benzo[e][1,4]diazepin-3-yl)-amide;
1-(2-Oxo-5-phenyl-2,3-dihydro-1H-benzo[e][1,4]diazepin-3-yl)-3-(4-phenoxy-phenyl)-urea.

In the above enantiomers the R or S assignment refers to the chiral carbon atom at the 3 position of the benzodiazepine core in formula (I) as defined above.

Typically the benzodiazepine derivative of formula (I) is the S enantiomer of any of the above-mentioned compounds.

The process of the present invention further provides the step of formulating a benzodiazepine derivative of formula (I) as defined above or a pharmaceutically acceptable salt thereof together with a pharmaceutically acceptable carrier or diluent, to yield a pharmaceutical preparation, such as a solid, liquid, suspension, emulsion or solution for injection.

Solid oral forms, for example may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

Certain benzodiazepine derivatives that are intermediates in the process of the present invention are novel. Accordingly, the present invention further provides a compound of formula (II): 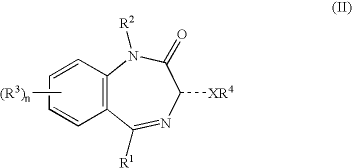
wherein z,4 , R¹, R², R³, R⁴, n and X are as defined above. Also provided by the present invention is a compound of formula (IIa): 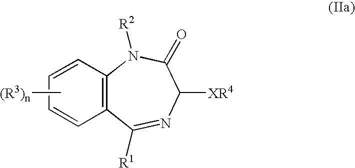
wherein R¹, R², R³, R⁴, n and X are as defined above.

The following Examples illustrate the invention.

### EXAMPLE 1

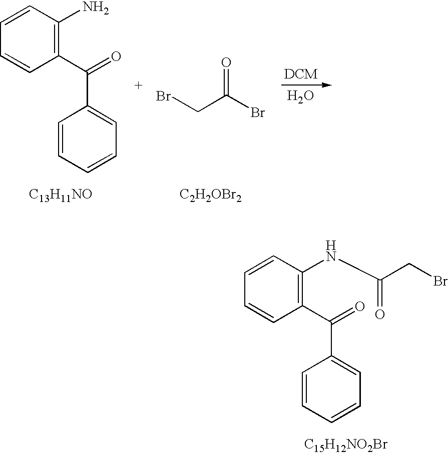

A 10 L flask was charged with 2-aminobenzophenone (444 g, 2.25 mol), dichlormethane (3500 ml) and water (250 ml). The reaction was cooled to 0° C. and bromoacetylbromide (500 g, 2.48 mol) was added dropwise maintaining the temperature below 5° C. The reaction was warmed to room temperature and stirred overnight. The aqueous layer was separated, the organic layer washed with water (2×2000 ml) and dried (MgSO₄). The mixture was filtered and the dichloromethane removed under reduced pressure to yield a yellow crystalline solid which was ground up and slurried in hexane (1600 ml). The product was filtered, washed with hexane (400 ml) and dried in a vacuum oven at room temperature overnight. Weight: 684 g, yield: 95%, confirmed by ¹H NMR.

### EXAMPLE 2

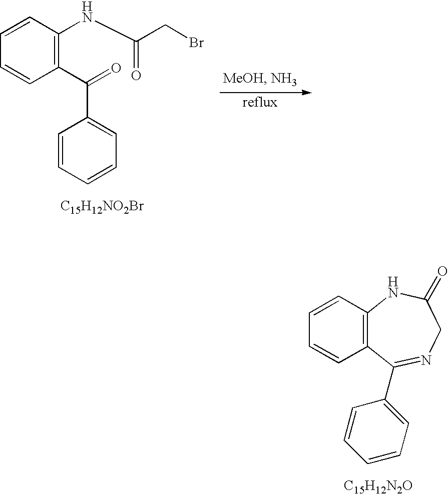

Methanol (6000 ml) was charged to a 10 L flask fitted with an IPA/dry ice condenser. Ammonia gas was added via subsurface addition over 7 h while the temperature was maintained at around 15° C. The addition was stopped and the reaction was left overnight. The addition of ammonia was continued for a further 2 h (7.26M solution). Stage 1 (300 g, 0.94 mol) was added and the reaction stirred at ˜18° C. for 30 min. TLC analysis showed that all starting material had been consumed. The reaction was heated at 50° C. for 2 h and then stirred overnight at room temperature. The volume of methanol was reduced to around 1300 ml. HPLC analysis: product 94.5%, by-product 4%. The methanol mixture was warmed to 40° C. and water (1300 ml) added. The reaction was left to stir at room temperature over the weekend. The slurry was filtered, the resultant solid washed with methanol/water 1:1 (3×300 ml) and dried in a vacuum oven at 50° C. overnight. Weight: 211 g, yield: 95%, chemical purity: 94.7%, confirmed by ¹H NMR.

### EXAMPLE 3

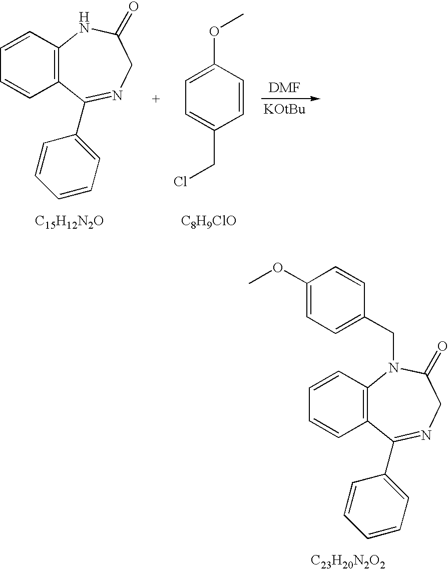

The product of Example 2 (30 g, 0.127 mol) and dimethylformamide (300 ml) were charged to 3-neck 1000 ml flask. Cooled to around 0° C. and KOtBu (16.4 g, 0.146 mol) added in one portion (slightly exothermic). 4-Methoxybenzylchloride (20 g, 0.127 mol), in dimethylformamide (40 ml) was added dropwise and the reaction stirred at room temperature for 1 h. TLC analysis indicated that all starting material had been consumed. Acetic acid (2 ml) was added and the dimethylformamide removed at 50° C. The residue was dissolved in toluene (600 ml) and washed with water (2×200 ml). The volume of toluene was reduced to around 200 ml and resulting solution added to rapidly stirred hexane (1000 ml). The solid was filtered, washed with hexane (500 ml) and dried in a vacuum oven at room temperature. Weight: 39 g, yield: 87%, HPLC purity: 95.4%.

### EXAMPLE 4

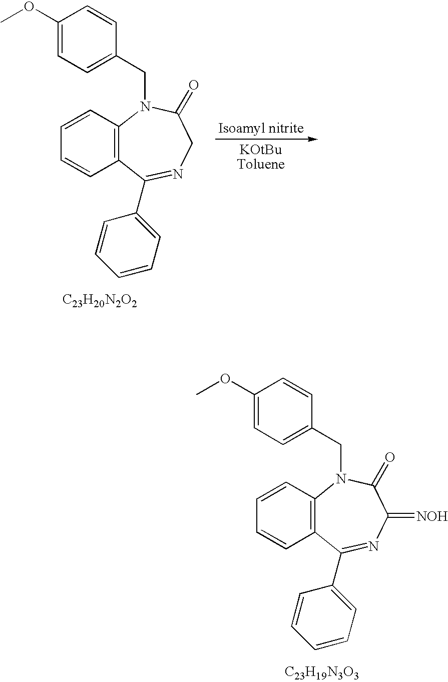

The product of Example 3 (40 g, 0.11 mol) and toluene (800 ml) were charged to a 2 L flange flask. The mixture was cooled to −20° C., KOtBu (30.26 g, 0.27 mol) added and stirred at −10° C. for 15 min. Isoamyl nitrite (15.77 g, 0.13 mol) was added and the reaction was stirred at −5° C. for 30 min. TLC analysis showed that all the starting material had been consumed. The mixture was poured onto water (1600 ml), ethyl acetate (1600 ml) and acetic acid (80 ml) and stirred for 10 min. The organic layer was separated and the aqueous fraction extracted with ethyl acetate (1000 ml). The organic layers were combined and washed with water (1000 ml). The volume of solvent was reduced to 500 ml, toluene (1000 ml) added and volume again reduced to around 500 ml. This procedure was repeated twice to remove all traces of ethyl acetate, water and acetic acid until a final volume of around 150-200 ml had been reached. The slurry was cooled in ice/water for 1 h, filtered and washed with cold toluene (2×100 ml) to yield a yellow solid which was dried in vacuum oven at 30° C. for 2 h. Weight: 33 g, yield: 76%, chemical purity: 99.4%, confirmed by ¹H NMR.

### EXAMPLE 5

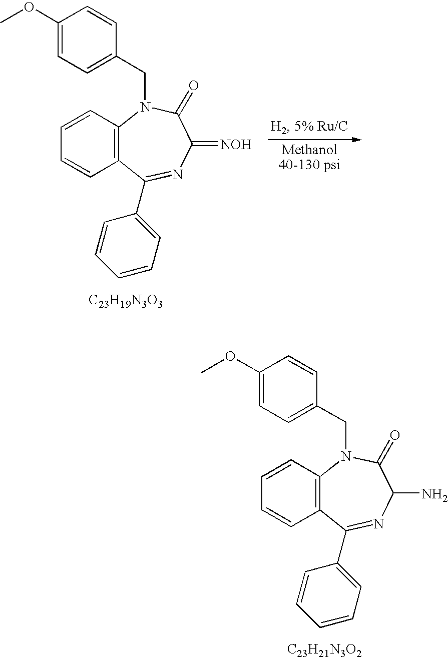

5% Ru/C (2.5 g) in methanol (50 ml) was charged to the hydrogenator. Stage 4 (10 g) in methanol (100 ml) was added and the slurry was heated at around 64° C., 40 psi of H₂, overnight with stirring. HPLC analysis showed that none of the starting material had been consumed. The reaction was heated at 70° C. and 40 psi of H₂ for 3 h. HPLC analysis showed starting material—69.2%, product—28.4% and impurity—0.8%. The pressure was increased to 130 psi of H₂ and the reaction heated overnight at 70° C. HPLC analysis showed product—92.6% and major impurity—3.2%. The reaction mixture was filtered through hyflo supercel and the catalyst washed with methanol (100 ml). The solvent was removed in vacuo to yield an orange oil which was dissolved in toluene (300 ml). The solvent was reduced in volume (around 100 ml) and poured onto rapidly stirred hexane (400 ml). The precipitate was filtered, washed with hexane (50 ml) and dried in vacuum oven at 30° C. Weight: 6 g, yield: 62%, chemical purity: 93%, confirmed by ¹H NMR. The filtrates were reduced in volume and the procedure repeated to yield a further 1.8 g of material with similar chemical purity. Overall yield: 81%.

### EXAMPLE 6

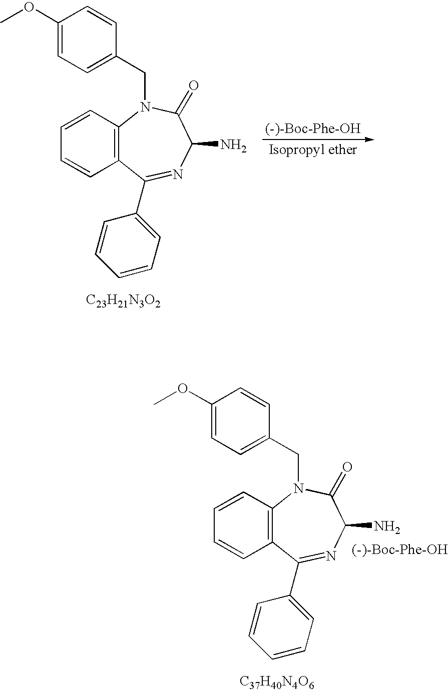

The product of Example 5 (106 mg) and (−) Boc-phenyl (38 mg, 0.5 eqivalents) were dissolved in dichloromethane. This solution was evaporated giving a pink foam to which water (20 mg) was added. Diisopropyl ether was then added until a solution was formed. The solvent was then left to evaporate over 18 hours leading to the formation of a solid. This material was then dissolved in the minimum volume of hot toluene and was then left to cool. This gave a crystalline solid which was collected by filtration (25 mg). Chiral HPLC analysis of this material showed an enantiomeric excess (S:R) of 86%. This material was then used as a source of seed crystals in the following dynamic kinetic resolutions.

### EXAMPLE 7

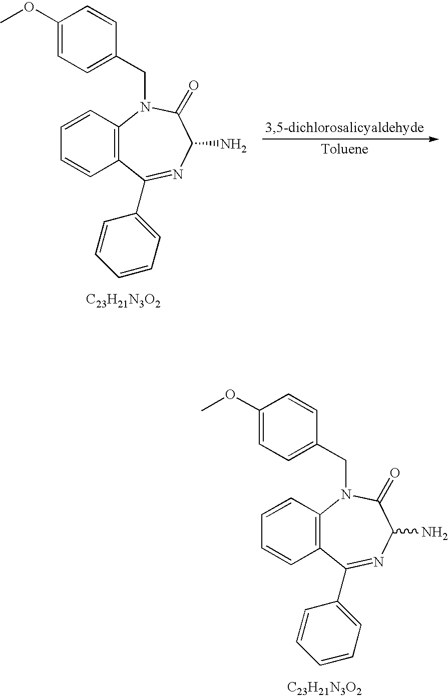

To a 100 ml flask, was charged the undesired (R) enantiomer isolated from the solution of Example 6 (9.9 g), toluene (55 ml) and 3,5-dichlorosalicylaldehyde (205 mg, 0.04 equiv.). The mixture was heated to achieve solution and stirred at room temperature under nitrogen overnight. A solid precipitated out. Solvent was removed in vacuo. A yellow solid was obtained. HPLC showed this solid was a mixture of two isomers at the ratio of 49.48%:43.62%.

### EXAMPLE 8

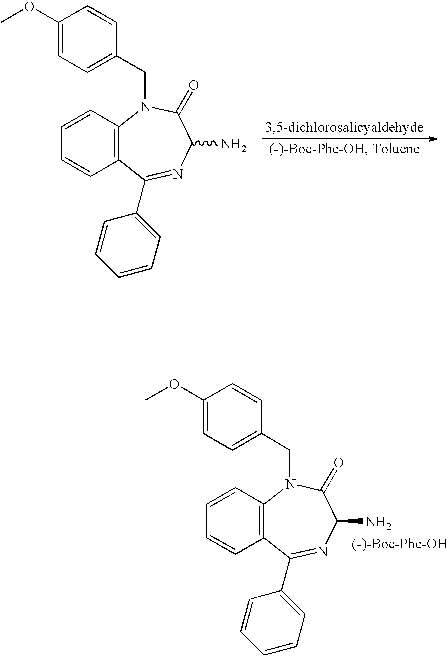 Reaction 1

To a 250 ml three-necked flask, was charged racemic product of Example 7 (9.9 g, racemized with 205 mg 3,5-dichlorosalicylaldehyde from 9.9 g unwanted isomer of Example 5) and toluene (66 ml). Charged (−)-Boc-Phe-OH (7.08 g, 1 equiv.) and heated to achieve solution. Water (0.2 ml, 0.46 equiv.) and a seed crystal were added and the solution stirred overnight at room temperature. The thick slurry was filtered, washed with toluene until yellow colour was removed and dried in the oven. Weight: 4.7 g, chiral purity: 99.7% ee. The mother liquor was concentrated in vacuo and the residue was dissolved in toluene (50 ml). Water (0.5 ml, 1.16 equiv.) and seed crystal were added. The solution was stirred at room temperature overnight. The thick slurry was filtered, washed with toluene and dried in an oven. Weight: 8.3 g, chiral purity: 99.8% ee. The above procedure was repeated to obtain the third crop of crystallisation product (1.2 g, 99.3% ee.). Overall weight: 14.2 g, overall yield: 84%.

Reaction 2

To a 250 ml three-necked flask, was charged racemic product of Example 5 (10.2 g, 87% pure by HPLC), (−)-Boc-Phe-OH (6.34 g, 1 equiv.) and toluene (60 ml). The mixture was heated to achive solution. Charged 3,5-dichlorosalicylaldehyde (183 mg, 0.04 equiv.) and stirred at room temperature for 30 mins. Water (0.43 ml, 1 equiv.) and a seed crystal were added. A thick slurry formed which was left standing over the weekend. The solid was filtered, washed with toluene and dried. Weight: 9.2 g, yield: 60.5%, chiral purity: 99.4% ee. HPLC chemical purity showed only two peaks: (−)-Boc-phe-OH and stage 5. The impurity in the starting material has been removed. This showed that the crystallisation achieved both high chiral purity and chemical purity. The mother liquor was concentrated in vacuo and dissolved in toluene (50 ml). Water (0.5 ml, 1.16 equiv.) and a seed crystal were added. A thick slurry formed which was left standing overnight. The solid was filtered, washed with toluene and dried. Weight: 1.6 g, yield: 10.5%, chiral purity: 99.8%. Overall yield: 70.9%.

### EXAMPLE 9

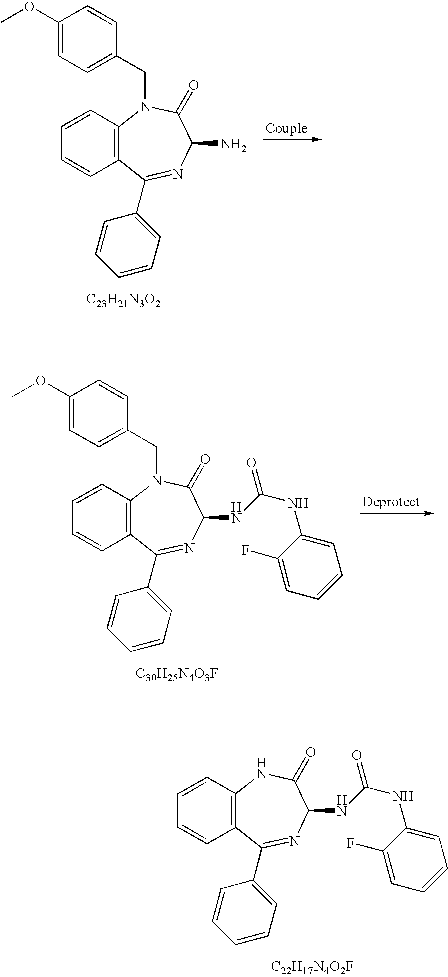

3(S)-amino-1,3-dihydro-1-(4-methoxyphenyl)-5-phenyl-2H-1,4-benzodiazepin-2-one)-amino (10 g) and THF (100 ml) were charged to a 250 ml three neck flask. Triethylamine (3.3 ml) was added and stirred for 30 min. 2-fluorophenylisocyanate (2.37 g) was added and the solution stirred at room temperature for 2 h. TLC analysis showed all starting material had been consumed. The solvent was removed in vacuo, the residue taken up in DCM (100 ml) and washed with water (100 ml and 2×50 ml). The DCM was dried (MgSO₄), filtered and concentrated in vacuo to yield a foam like solid. ¹H NMR spectrum showed that Boc-phe-OH was still present and therefore the crude material was dissolved in ethyl acetate (150 ml) and washed with NaHCO₃. The organic layer was dried (MgSO₄), filtered and solvent removed in vacuo to yield a white solid. The material was used in the next stage without further purification.

Deprotection

3(S)-(2-fluorophenylureyl)-1,3-dihydro-1-(4-methoxyphenyl)-5-phenyl-2H-1,4-benzodiazepin-2-one)-amino Stage 7 (9 g) was dissolved in anisole (40 ml) and cooled to 0° C. AlCl₃ (21 g) was added in one portion and the solution stirred at room temperature over the weekend. TLC analysis showed that only a trace of starting material was remaining. Dichloromethane (200 ml) was added, cooled (ice bath) and water (200 ml) added. The aqueous layer was extracted with dichloromethane (2×150 ml), the organic layers combined, washed with water (2×200 ml) and dried (MgSO₄). After filtration the solvents were removed in vacuo and the residue slurried in isopropanol (20 ml) and hexane (40 ml). The product was filtered, washed with hexane and dried. Weight: 5.6 g, yield: 91% over two steps, chemical purity: 96.4%, chiral purity: 99.9%.

1. A process for producing a compound which is a benzodiazepine derivative of formula (I): 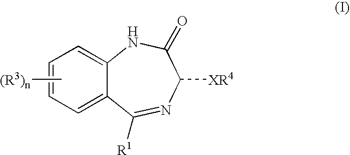
wherein: [symbol]
represents [symbol] or [symbol]
R¹ represents C₁₋₆ alkyl, aryl or heteroaryl;
each R³ is the same or different and represents halogen, hydroxy, C₁₋₆ alkyl, C₁₋₆ alkoxy, C₁₋₆ alkylthio, C₁₋₆ haloalkyl, C₁₋₆ haloalkoxy, amino, mono(C_{1 6} alkyl)amino, di(C₁₋₆ alkyl)amino, nitro, cyano, —CO₂R′, —CONR′R″, —NH—CO—R″, —S(O)R″, —S(O)₂R′, —NH—S(O)₂R′, —S(O)NR′R″ or —S(O)2NR′R″, wherein each R′ and R″ is the same or different and represents hydrogen or C₁₋₆ alkyl;
n is from 0 to 3;
X represents —NH—, —N(C₁-C₆ alkyl)-, —CO—, —CO—NR′—, —S(O)— or —S(O)₂—, wherein R′ is hydrogen or a C₁-C₆ alkyl group; and
R⁴ represents hydrogen; or —CO—R₄′ or —CO—NH—R⁴′, wherein R⁴′is a C₁-C₆ alkyl, C₁-C₆ hydroxyalkyl, aryl, heteroaryl, carbocyclyl or heterocyclyl group, which group is substituted by a C₁-C₆ hydroxyalkyl, aryl, heteroaryl, carbocyclyl or heterocyclyl group or a —(C₁-C₄ alkyl)-X₁—(C₁-C₄ alkyl)-X₂—(C₁-C₄ alkyl) group, wherein X₁ represents —O—, —S— or —NR′—, wherein R′ represents H or a C₁-C₄ alkyl group and X₂ represents —CO—, —SO— or —SO₂—; or R⁴′ represents -A₁-Y-A₂, wherein:
A₁ is an aryl, heteroaryl, carbocyclyl or heterocyclyl group;
Y represents a direct bond or a C₁-C₄ alkylene, —SO₂—, —CO—, —O—, —S or —NR′—,
wherein R′ is a C₁-C₆ alkyl group; and
A₂ is an aryl, heteroaryl, carbocyclyl or heterocyclyl group;
or R⁴ is a group selected from aryl-C(O)—C(O)—, heteroaryl-C(O)—C(O)—, carbocyclyl-C(O)—C(O)—, heterocyclyl-C(O)—C(O)— and -ZR⁵, wherein:
Z represents —CO—, —S(O)— or —S(O)₂—; and
R⁵ represents C₁-₆ alkyl, hydroxy, C₁-₆ alkoxy, C₁-₆ alkylthio, aryl, heteroaryl, carbocyclyl, heterocyclyl, aryl-(C₁-6 alkyl)-, heteroaryl-(C₁-₆ alkyl)-, carbocyclyl-(C₁-₆ alkyl)-, heterocyclyl-(C₁-₆ alkyl)-, aryl-(C₁-₆ alkyl)-O—, heteroaryl-(C₁-₆ alkyl)-O—, carbocyclyl-(C₁-₆ alkyl)-O—, heterocyclyl-(C₁-₆ alkyl)-O— or —NR′R″ wherein each R′ and R″ is the same or different and represents hydrogen, C₁-₆ alkyl, carbocyclyl, heterocyclyl, aryl, heteroaryl, aryl-(C₁-₆ alkyl)-, heteroaryl-(C₁-₆ alkyl)-, carbocyclyl-(C₁-₆ alkyl)— or heterocyclyl-(C₁-₆ alkyl)-; or a pharmaceutically acceptable salt thereof; which process comprises:
(a) subjecting a racemic benzodiazepine derivative of formula (IIa): 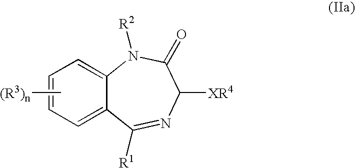
wherein R¹, R³, R⁴, n and X are as defined above, and R represents an amino protecting group, to crystallisation induced dynamic resolution to yield a benzodiazepine derivative of formula (II): 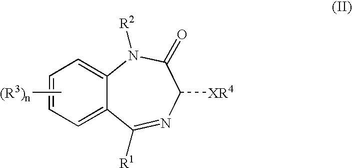
wherein [symbol] , R¹, R², R³, R⁴, n and X are as defined above, and
(b) deprotecting the benzodiazepine derivative of formula (II) as defined above to yield a benzodiazepine derivative of formula (I) or a pharmaceutically acceptable form thereof as defined above.

2. A process according to claim 1 wherein the amino protecting group is a group —(CH₂)ₘ—R′, wherein m is 0 or an integer of from 1 to 3 and R′ is a group —O—(C₁-C₆ alkyl), —C(O)O—(C₁-C₆ alkyl), —OC(O)—(C₁-C₆ alkyl), aryl, heteroaryl, carbocyclyl or heterocyclyl.

3. A process according to claim 1, wherein the moiety XR⁴ in formula (II) is sensitive to the conditions of deprotection of step (b), which process further comprises, prior to the deprotection step (b), converting the said moiety [symbol] XR⁴ into another moiety of formula [symbol] XR⁴ which is not sensitive to the conditions of deprotection.

4. A process according to claim 1, which process further comprises:
(c) converting the moiety [symbol] XR⁴ in the benzodiazepine derivative of formula (I), which moiety is not sensitive to the conditions of deprotection used in the preceding step (b), into another moiety [symbol] XR⁴ which is either insensitive or sensitive to the conditions of deprotection used in step (b).

5. A process according to claim 4, wherein, in step (c), [symbol] XR⁴ is an amine (—NH₂) which is converted to a 2-fluorophenylurea (—NHC(O)NH-(2F-Ph)) group.

6. A process according to claim 1 which further comprises producing the racemic benzodiazepine derivative of formula (IIa) by a process which comprises:
reducing a compound of formula (III): 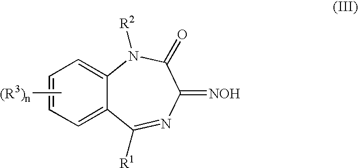
wherein R¹, R², R³ and n are as defined in claim 1 using hydrogen gas and a reducing catalyst in an inert solvent, to produce the desired compound of formula (IIa).

7. A process according to claim 6, which further comprises producing the compound of formula (III) as defined in claim 6 by treating a compound of formula (IV): 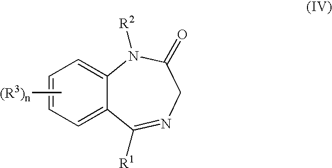
wherein R¹, R², R³ and n are as defined in claim 6, with isoamyl nitrite and a base in an inert solvent.

8. A process according to claim 1 wherein the bezodiazepine derivative of formula (I) is the R enantiomer or the S enantiomer of:
(a) one of the following:
N-(2-Oxo-5-phenyl-2,3-dihydro-1H-benzo[e][1,4]diazepin-3-yl)-acetamide;
1,1-Diethyl-3-(2-oxo-5-phenyl-2,3-dihydro-1H-benzo[e][1,4]diazepin-3-yl)-urea;
N-(2-Oxo-5-phenyl-2,3-dihydro-1H-benzo[e][1,4]diazepin-3-yl)-propionamide;
N-(2-Oxo-5-phenyl-2,3-dihydro-1H-benzo[e][1,4]diazepin-3-yl)-butyramide;
N-(2-Oxo-5-phenyl-2,3-dihydro-1H-benzo[e][1,4]diazepin-3-yl)-isobutyramide;
2,2-Dimethyl-N-(2-Oxo-5-phenyl-2,3-dihydro-1H-benzo[e][1,4]diazepin-3-yl)-propionamide;
Cyclopentanecarboxylic acid (2-oxo-5-phenyl-2,3-dihydro-1H-benzo[e][1,4]diazepin-3-yl)-amide;
Cyclohexanecarboxylic acid 2-oxo-5-phenyl-2,3-dihydro-1H-benzo[e][1,4]diazepin-3-yl)-amide;
3-MethoxyN-(2-oxo-5-phenyl-2,3-dihydro-1H-benzo[e][1,4]diazepin-3-yl)-benzamide;
4-MethoxyN-(2-oxo-5-phenyl-2,3-dihydro-1H-benzo[e][1,4]diazepin-3-yl)-benzamide;
2-MethoxyN-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)-3-trifluoromethyl-benzamide;
N-(2-Oxo-5-phenyl-2,3-dihydro-1H-benzo[e][1,4]diazepin-3-yl)-benzamide;
Thiophene-2-carboxylic acid (2-oxo-5-phenyl-2,3-dihydro-1H-benzo[e][1,4]diazepin-3-yl)-3-amide;
Furan-2-carboxylic acid (2-oxo-5-phenyl-2,3-dihydro-1H-benzo[e][1,4]diazepin-3-yl)-amide;
Piperidine-1-carboxylic acid (2-oxo-5-phenyl-2,3-dihydro-1H-benzo[e][1,4]diazepin-3-yl)-amide;
Morpholine-4-carboxylic acid (2-oxo-5-phenyl-2,3-dihydro-1H-benzo[e][1,4]diazepin-3-yl)-amide;
4-Nitro-N-(2-oxo-5-phenyl-2,3-dihydro-1H-benzo[e][1,4]diazepin-3-yl)-benzamide;
3-Nitro-N-(2-oxo-5-phenyl-2,3-dihydro-1H-benzo[e][1!,4]diazepin-3-yl)-benzamide;
4-Methyl-piperazine-1-carboxylic acid-(2-oxo-5-phenyl-2,3-dihydro-1H-benzo[e][1,4]diazepin-3-yl)-amide;
3,4-Dichloro-N-(2-oxo-5-phenyl-2,3-dihydro-1H-benzo[e][1,4]diazepin-3-yl)-benzamide;
N-(2-oxo-5-phenyl-2,3-dihydro-1H-benzo[e][1,4]diazepin-3-yl)-2-trifluoromethyl-benzamide;
4-Bromo-N-(2-oxo-5-phenyl-2,3-dihydro-1H-benzo[e][1,4]diazepin-3-yl)-benzamide;
2-Methyl-N-(2-oxo-5-phenyl-2,3-dihydro-1H-benzo[e][1,4]diazepin-3-yl)-benzamide;
2-Chloro-N-(2-oxo-5-phenyl-2,3-dihydro-1H-benzo[e][1,4]diazepin-3-yl)-benzamide;
2-Nitro-N-(2-oxo-5-phenyl-2,3-dihydro-1H-benzo[e][1,4]diazepin-3-yl)-benzamide;
2-Methoxy-4-nitro-N-(2-oxo-5-phenyl-2,3-dihydro-1H-benzo[e][1,4]diazepin-3-yl)-benzamide;
(S)-2-Methoxy-4-nitro-N-(2-oxo-5-phenyl-2,3-dihydro-1H-benzo[e][1,4]diazepin-3-yl)-benzamide;
Benzo[b]thiophene-3-carboxylic acid (2-oxo-5-phenyl-2,3-dihydro-1H-benzo[e][1,4]diazepin-3-yl)-amide;
2,3-Dihydro-benzofuran-5-carboxylic acid (2-oxo-5-phenyl-2,3-dihydro-1H-benzo[e][1,4]diazepin-3-yl)-amide;
Isoxazole-5-carboxylic acid (2-oxo-5-phenyl-2,3-dihydro-1H-benzo[e][1,4]diazepin-3-yl)-amide;
Benzo[b]thiophene-2-carboxylic acid (2-oxo-5-phenyl-2,3-dihydro-1H-benzo[e][1,4]diazepin-3-yl)-amide;
Thiophen-3-carboxylic acid (2-oxo-5-phenyl-2,3-dihydro-1H-benzo[e][1,4]diazepin-3-yl)-amide;
N-(2-Oxo-5-phenyl-2,3-dihydro-1H-benzo[e][1,4]diazepin-3-yl)-isonicotmaniide;
N-(2-Oxo-5-phenyl-2,3-dihydro-1H-benzo[e][1,4]diazepin-3-yl)-nicotinamide;
N-(2-Oxo-5-phenyl-2,3-dihydro-1H-benzo[e][1,4]diazepin-3-yl)-methanesulfonamide;
Propane-1-sulfonic acid-(2-oxo-5-phenyl-2,3-dihydro-1H-benzo[e][1,4]diazepin-3-yl)-amide;
Butane-1-sulfonic acid-(2-oxo-5-phenyl-2,3-dihydro-1H-benzo[e][1,4]diazepin-3-yl)-amide;
2-Bromo-N-(2-oxo-5-phenyl-2,3-dihydro-1H-benzo[e][1,4]diazepin-3-yl)-benzenesulfonamide;
3-Bromo-N-(2-oxo-5-phenyl-2,3-dihydro-1H-benzo[e][1,4]diazepin-3-yl)-benzenesulfonamide;
4-Bromo-N-(2-oxo-5-phenyl-2,3-dihydro-1H-benzo[e][1,4]diazepin-3-yl)-benzenesulfonamide;
2-Fluoro-N-(2-oxo-5-phenyl-2,3-dihydro-1H-benzo[e][1,4]diazepin-3-yl)-benzenesulfonamide;
3-(2-Nitro-benzylamino)-5-phenyl-1,3-dihydro-benzo[e][1,4]diazepin-2-one;
3-(3-Nitro-benzylamino)-5-phenyl-1,3-dihydro-benzo[e][1,4]diazepin-2-one;
3-(4-Nitro-benzylamino)-5-phenyl-1,3-dihydro-benzo[e][1,4]diazepin-2-one;
3-(2-Methoxy-benzylamino)-5-phenyl-1,3-dihydro-benzo[e][1,4]diazepin-2-one;
3-(3-Methoxy-benzylamino)-5-phenyl-1,3-dihydro-benzo[e][1,4]diazepin-2-one;
5-Phenyl-3-(2-trifluoromethyl-benzylammo)-1,3-dihydro-benzo[e][1,4]diazepin-2-one;
5-Phenyl-3-(3-trifluoromethyl-benzylamino)-1,3-dihydro-benzo[e][1,4]diazepin-2-one;
5-Phenyl-3-(4-trifluoromethyl-benzylamino)-1,3-dihydro-benzo[e][1,4]diazepin-2-one;
3-[(Furan-2-ylmethyl)-amino]-5-phenyl-1,3-dihydro-benzo[e][1,4]diazepin-2-one;
N-(7-Chloro-2-oxo-S-phenyl-2,3-dihydro-1H-benzo[e][1,4]diazepin-3-yl-acetamide;
N-(7-Chloro-2-oxo-5-phenyl-2,3-dihydro-1H-benzo[e][1,4]diazepin-3-yl)-isobutyramide;
N-(7-Chloro-2-oxo-5-phenyl-2,3-dihydro-1H-benzo[e][1,4]diazepin-3-yl)-methanesulfonamide;
Furan-2-carboxylic acid (7-chloro-2-oxo-5-phenyl-2,3-dihydro-1H-benzo[e][1,4]diazepin-3-yl)-amide;
Thiophene-2-carboxylic acid (7-chloro-2-oxo-5-phenyl-2,3-dihydro-1H-benzo[e][1,4]diazepin-3-yl)-amide;
Cyclohexanecarboxylic acid (7-Chloro-2-oxo-5-phenyl-2,3-dihydro-1H-benzo[e][1,4]diazepin-3-yl)-amide;
N-(7-Chloro-2-oxo-5-phenyl-2,3-dihydro-1H-benzo[e][1,4]diazepin-3-yl)-2-methoxy-benzamide;
N-(7-Chloro-2-oxo-5-phenyl-2,3-dihydro-1H-benzo[e][1,4]diazepin-3-yl)-4-methoxy-benzamide;
N-(7-Chloro-2-oxo-5-phenyl-2,3-dihydro-1H-benzo[e][1,4]diazepin-3-yl)-2-nitro-benzamide;
2-(2-Methoxy-phenyl)N-(2-oxo-5-phenyl-2,3-dihydro-1H-benzo[e][1,4]diazepin-3-yl)-acetamide;
2-(3-Methoxy-phenyl)N-(2-oxo-5-phenyl-2,3-dihydro-1H-benzo[e][1,4]diazepin-3-yl)-acetamide;
2-(4-Methoxy-phenyl)N-(2-oxo-5-phenyl-2,3-dihydro-1H-benzo[e][1,4]diazepin-3-yl)-acetamide;
2-(4-Nitro-phenyl)N-(2-oxo-5-phenyl-2,3-dihydro-1H-benzo[e][1,4]diazepin-3-yl)-acetamide;
2-(3-Nitro-phenyl)N-(2-oxo-5-phenyl-2,3-dihydro-1H-benzo[e][1,4]diazepin-3-yl)-acetamide;
N-(2-Oxo-5-phenyl-2,3-dihydro-1H-benzo[e][1,4]diazepin-3-yl)-2-(2-txifluoromethyl-phenyl)-acetamide;
N-(2-Oxo-5-phenyl-2,3-dihydro-1H-benzo[e][1,4]diazepin-3-yl)-2-(3-trifluoromethyl-phenyl)-acetamide;
N-(2-Oxo-5-phenyl-2,3-dihydro-1H-benzo[e][1,4]diazepin-3-yl)-2-(4-trifluoromethyl-phenyl)-acetamide;
1-(2-Methoxy-phenyl)-3-(2-oxo-5-phenyl-2,3-dihydro-1H-benzo[e][1,4]diazepin-3-yl)-urea;
1-(2-Nitro-phenyl)-3-(2-oxo-5-phenyl-2,3-dihydro-1H-benzo[e][1,4]diazepin-3-yl)-urea;
1-(2-Chloro-phenyl)-3-(2-oxo-5-phenyl-2,3-dihydro-1H-benzo[e][1,4]diazepin-3yl)-urea;
1-(4-Chloro-phenyl)-3-(2-oxo-5-phenyl-2,3-dihydro-1H-benzo[e][1,4]diazepin-3-yl)-urea;
1-(2-oxo-5-phenyl-2,3-dihydro-1H-benzo[e][1,4]diazepin-3-yl)-3-p-tolyl-urea;
1-(2-Fluoro-phenyl)-3-(2-oxo-5-phenyl-2,3-dihydro-1H-benzo[e][1,4]diazepin-3yl)-urea;
1-(4-Fluoro-phenyl)-3-(2-oxo-5-phenyl-2,3-dihydro-1H-benzo[e][1,4]diazepin-3-yl)-urea;
4-Methanesulfonyl-2-methoxy-N-(2-oxo-5-phenyl-2,3-dihydro-1H-benzo[e][1,4]diazepin-3-yl)-benzamide;
5-Acetyl-2-ethoxy-N-(2-oxo-5-phenyl-2,3-dihydro-1H-benzo[e][1,4]diazepin-3-yl)-benzamide;
6-Fluoro-4H-benzo[1,3]dioxine-8-carboxylic acid (2-oxo-5-phenyl-2,3-dihydro-1H-benzo[e][1,4]diazepin-3-yl)-amide;
2-Methoxy-N-(2-oxo-5-phenyl-2,3-dihydro-1H-benzo[e][1,4]diazepin-3-yl)-4-trifluoromethyl-benzamide;
2,4,5-Trifluoro-N-(2-oxo-5-phenyl-2,3-dihydro-1H-benzo[e][1,4]diazepin-3-yl)-benzamide;
2-Hydroxy-N-(2-oxo-5-phenyl-2,3-dihydro-1H-benzo[e][1,4]diazepin-3-yl)-benzamide;
1H-Indole-7-carboxylic acid (2-oxo-5-phenyl-2,3-dihydro-1H-benzo[e][1,4]diazepin-3-yl)-amide;
3-Methoxy-naphthalene-2-carboxylic acid (2-oxo-5-phenyl-2,3-dihydro-1H-benzo[e][1,4]diazepin-3-yl)-amide;
N-[7-Chloro-5-(2-fluoro-phenyl)-2-oxo-2,3-dihydro-1H-benzo[e][1,4]diazepine-3-yl]-4-methoxoy-benzamide;
1-(2-Fluoro-benzyl)-3-(2-oxo-5-phenyl-2,3-dihydro-1H-benzo[e][1,4]diazepin-3-yl)-urea;
1-(4-Methoxy-benzyl)-3-(2-oxo-5-phenyl-2,3-dihydro-1H-benzo[e][1,4]diazepin-3-yl)-urea;
1-(3-Methyl-benzyl)-3-(2-oxo-5-phenyl-2,3-dihydro-1H-benzo[e][1,4]diazepin-3-yl)-urea;
1-(2-Oxo-5-phenyl-2,3-dihydro-1H-benzo[e][1,4]diazepin-3-yl)-3-(4-trifluoromethyl-phenyl)-urea;
4-Chloro-2-methoxy-N-(2-oxo-5-phenyl-2,3-dihydro-1H-benzo[e][1,4]diazepin-3-yl)-benzamide;
4-Methoxy-3-nitro-N-(2-oxo-5-phenyl-2,3-dihydro-1H-benzo[e][1:,4]diazepin-3-yl)benzamide;
3-Methoxy-2-nitro-N-(2-oxo-5-phenyl-2,3-dihydro-1H-benzo[e][1,4]diazepin-3-yl)-benzamide;
5-Chloro-2-methoxy-N-(2-oxo-5-phenyl-2,3-dihydro-1H-benzo[e][1,4]diazepin-3-yl)benzamide;
5-Fluoro-2-methoxy-N-(2-oxo-5-phenyl-2,3-dib.ydro-1H-benzo[e][1,4]diazepin-3-yl)-benzamide;
2-Methoxy-4-nitro-N-(2-oxo-5-phenyl-2,3-dihydro-1H-benzo[e][1,4]diazepin-3-yl)-benzamide;
5-Methoxy-2-nitro-N-(2-oxo-5-phenyl-2,3-dihydro-1H-benzo[e][1,4]diazepin-3-yl)-benzamide;
3-Methoxy-4-nitro-N-(2-oxo-5-phenyl-2,3-dihydro-1H-benzo[e][1,4]diazepin-3-yl)-benzamide;
3-(2-Methoxy-phenyl)-N-(2-oxo-5-phenyl-2,3-dihydro-1H-benzo[e][1,4]diazepin-3-yl)propionamide;
3-(3-Methoxy-phenyl)-N-(2-oxo-5-phenyl-2,3-dihydro-1H-benzo[e][1,4]diazepm-3-yl)-propionamide;
3-(4-Methoxy-phenyl)-N-(2-oxo-5-phenyl-2,3-dihydro-1H-benzo[e][1,4]diazepin-3-yl)-propionamide;
N-[5-(3-Chloro-phenyl)-2-oxo-2,3-dihydro-1H-benzo[e][1,4]diazepin-3-yl]-2-methoxy-benzamide;
N-[5-(3-Chloro-phenyl)-2-oxo-5-phenyl-2,3-dihydro-1H-benzo[e][1,4]diazepin-3-yl]-4-methoxy-benzamide;
N-[5-(3-Chloro-phenyl)-2-oxo-5-phenyl-2,3-dihydro-1H-benzo[e][1,4]diazepin-3-yl]-2-nitro-benzamide;
N-[5-(3-Chloro-phenyl)-2-oxo-5-phenyl-2,3-dihydro-1H-benzo[e][1,4]diazepin-3-yl]-4-nitro-benzamide;
4-Methoxy-N-[2-oxo-5-(4-trifluoromethyl-phenyl)-2,3-dihydro-1H-benzo[e][1,4]diazepin-3-yl]-benzamide;
2-Methoxy-N-[2-oxo-5-(3-trifluoromethyl-phenyl)-2,3-dihydro-1H-benzo[e][1,4]diazepin-3-yl]-benzamide;
4-Methoxy-N-[2-oxo-5-(3-trifluoromethyl-phenyl)-2,3-dihydro-1H-benzo[e][1,4]diazepin-3-yl]-benzamide;
2-Ethoxy-N-(2-oxo-5-phenyl-2,3-dihydro-1H-benzo[e][1,4]diazepin-3-yl)-benzamide;
2,4-Dimethoxy-N-(2-oxo-5-phenyl-2,3-dihydro-1H-benzo[e][1,4]diazepin-3-yl)-benzamide;
2-Bromo-5-methoxy-N-(2-oxo-5-phenyl-2,3-dihydro-1H-benzo[e][1,4]diazepin-3-yl)-benzamide;
2-Methoxy-N-[5-(3-mehtoxy-phenyl)-2-oxo-2,3-dihydro-1H-benzo[e][1,4]diazepin-3-yl]-benzamide N-[5-(3-Methoxy-phenyl)-2-oxo-5-phenyl-2,3-dihydro-1H-benzo[e][1,4]diazepin-3-yl]-4-nitro-benzamide;
2-Methoxy-N-(8-methyl-2-oxo-5-phenyl-2,3-dihydro-1H-benzo[e][1,4]diazepin-3-yl)-benzamide;
2-Chloro-4-methanesulfonyl-N-(2-oxo-5-phenyl-2,3-dihydro-1H-benzo[e][1,4]diazepin-3-yl)-benzamide;
2-Dimethylamino-N-(2-oxo-5-phenyl-2,3-dihydro-1H-benzo[e][1,4]diazepin-3-yl)-benzamide;
(2-Oxo-5-phenyl-2,3-dihydro-1H-benzo[e][1,4]diazepin-3-yl)-carbamic acid benzyl ester; 1-(3,5-Dimethyl-phenyl)-3-(2-oxo-5-phenyl-2,3-dihydro-1H-benzo[e][1,4]diazepin-3-yl)-urea;
1-(2-Oxo-5-phenyl-2,3-dihydro-1H-beπzo[e][1,4]diazepin-3-yl)-3-(4-trifluoromethoxy-phenyl)-urea;
1-(4-Bromo-2-trifluoromethyl-phenyl)-3-(2-oxo-5-phenyl-2,3-dihydro-1H-benzo[e][1,4]diazepin-3-yl)-urea;
1-(4-Bromo-bezzyl)-3-(2-oxo-5-phenyl-2,3-dihydro-1H-benzo[e][1,4]diazepin-3-yl)-urea;
1-(2,3-Dichloro-phenyl)-3-(2-oxo-5-phenyl-2,3-dihydro-1H-benzo[e][1,4]diazepin-3-yl)-urea;
1-(2,6-Dimethyl-phenyl)-3-(2-oxo-5-phenyl-2,3-dihydro-1H-benzo[e][1,4]diazepin-3-yl)-urea;
1-(2-Chloro-6-methyl-phenyl)-3-(2-oxo-5-phenyl-2,3-dihydro-1H-benzo[e][1,4]diazepin-3-yl)-urea;
1-(4-Nitro-phenyl)-3-(2-oxo-5-phenyl-2,3-dihydro-1H-benzo[e][1,4]diazepin-3-yl)-urea;
1-(2-Methylsulfanyl-phenyl)-3-(2-oxo-5-phenyl-2,3-dihydro-1H-benzo[e][1,4]diazepin-3-yl)-urea;
1-(2,6-Dichloro-phenyl)-3-(2-oxo-5-phenyl-2,3-dihydro-1H-benzo[e][1,4]diazepin-3-yl)-urea;
5-tert-Butyl-2-methoxy-N-(2-oxo-5-phenyl-2,3-dihydro-1H-benzo[e][1,4]diazepin-3-yl)-benzamide;
2,5-Dimethoxy-N-(2-oxo-5-phenyl-2,3-dihydro-1H-benzo[e][1,4]diazepin-3-yl)-benzamide;
1-(2,6-Difluoro-phenyl)-3-(2-oxo-5-phenyl-2,3-dihydro-1H-benzo[e][1,4]diazepin-3-yl)-urea;
1-(3-Fluoro-phenyl)-3-(2-oxo-5-phenyl-2,3-dihydro-1H-benzo[e][1,4]diazepin-3-yl)-urea;
1-(3-Methoxy-phenyl)-3-(2-oxo-5-phenyl-2,3-dihydro-1H-benzo[e][1,4]diazepin-3-yl)-urea;
1-(2-Oxo-5-phenyl-2,3-dihydro-1H-benzo[e][1,4]diazepin-3-yl)-3-(3-trifluoromethyl-phenyl)-urea;
1-(3-Chloro-phenyl)-3-(2-oxo-5-phenyl-2,3-dihydro-1H-benzo[e][1,4]diazepin-3-yl)-urea;
2-Methoxy-4-methylsulfanyl-N-(2-oxo-5-phenyl-2,3-dihydro-1H-benzo[e][1,4]diazepin-3-yl)-benzamide;
4-Methanesulfonyl-N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)terephthalamic acid methyl ester;
2-Fluoro-N-(2-oxo-5-phenyl-2,3-dihydro-1H-benzo[e][1,4]diazepin-3-yl)-benzamide;
2,6-Difluoro-N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)-2-propoxy-benzamide;
2-Iodo-N-(2-oxo-5-phenyl-2,3-dihydro-1H-benzo[e][1,4]diazepin-3-yl)-benzamide;
3-Methoxy-N-(2-oxo-5-phenyl-2,3-dihydro-1H-benzo[e][1,4]diazepin-3-yl)-terephthalamic acid methyl ester;
4-Amino-5-chloro-2-methoxy-N-(2-oxo-5-phenyl-2,3-dihydro-1H-benzo[e][1,4]diazepin-3-yl)-benzamide;
1-(2-Oxo-5-phenyl-2,3-dihydro-1H-benzo[e][1,4]diazepin-3-yl)-3-m-tolyl-urea;
2-Methylsulfanyl-N-(2-oxo-5-phenyl-2,3-dihydro-1H-benzo[e][1,4]diazepin-3-yl)-benzamide;
2-Methoxy-N-(2-oxo-5-phenyl-2,3-dihydro-1H-benzo[e][1,4]diazepin-3-yl)-5-sulfamoyl-benzamide;
2-Hydroxy-N-(2-oxo-5-phenyl-2,3-dihydro-1H-benzo[e][1,4]diazepin-3-yl)-3-phenyl-propionamide;
3-Hydroxy-N-(2-oxo-5-phenyl-2,3-dihydro-1H-benzo[e][1,4]diazepin-3-yl)-3-phenyl-propionamide;
3-(2-Fluoro-phenyl)-1-methyl-1-(2-oxo-5-phenyl-2,3-dihydro-1H-benzo[e][1,4]diazepin-3-yl)-urea;
2-Methoxy-N-methyl-4-nitro-N-(2-oxo-5-phenyl-2,3-dihydro-1H-benzo[e][1,4]diazepin-3-yl)-benzamide;
1-tert-Butyl-3-(2-oxo-5-phenyl-2,3-dihydro-1H-benzo[e][1,4]diazepin-3-yl)-urea;
1-Cycloheyl-3-(2-oxo-5-phenyl-2,3-dihydro-1H-benzo[e][1,4]diazepin-3-yl)-urea;
1-Ethyl-3-(2-oxo-5-phenyl-2,3-dihydro-1H-benzo[e][1,4]diazepin-3-yl)-urea;
1-Butyl-3-(2-oxo-5-phenyl-2,3-dihydro-1H-benzo[e][1,4]diazepin-3-yl)-urea;
4,5-Dimethyl-furan-2-carboxylic acid (2-oxo-5-phenyl-2,3-dihydro-1H-benzo[e][1,4]diazepin-3-yl)amide;
Piperidine-1-carboxylic acid (7-chloro-2-oxo-5-phenyl-2,3-dihydro-1H-benzo[e][1,4]diazepin-3-yl)-amide;
N-[5-(3-Chloro-phenyl)-2-oxo-5-phenyl-2,3-dihydro-1H-benzo[e][1,4]diazepin-3-yl)acetamide;
N-[5-(3-Chloro-phenyl)-2-oxo-5-phenyl-2,3-dihydro-1H-benzo[e][1,4]diazepin-3-yl]-isobutyramide;
Furan-2-carboxylic acid[5-(3-chloro-phenyl)-2-oxo-5-phenyl-2,3-dihydro-1H-benzo[e][1,4]diazepin-3-yl]-amide;
Thiophene-2-carboxylic acid[5-(3-chloro-phenyl)-2-oxo-5-phenyl-2,3-dihydro-1H-benzo[e][1,4]diazepin-3-yl]-amide;
Cyclohexanecarboxylic acid[5-(3chloro-phenyl)-2-oxo-5-phenyl-2,3-dihydro-1H-benzo[e][1,4]diazepin-3-yl]-amide;
Piperidine-1-carboxylic acid[5-(3-chloro-phenyl)-2-oxo-5-phenyl-2,3-dihydro-1H-benzo[e][1,4]diazepin-3-yl]-amide;
N-[5-(3-Chloro-phenyl)-2-oxo-5-phenyl-2,3-dihydro-1H-benzo[e][1,4]diazepin-3-yl]isonicotinamide;
5-Methyl-furan-2-carboxylic acid (2-oxo-5-phenyl-2,3-dihydro-1H-benzo[e][1,4]diazepin-3-yl)-amide;
Pyrazine-2-carboxylic acid (2-oxo-5-phenyl-2,3-dihydro-1H-benzo[e][1,4]diazepin-3-yl)-amide;
N-[5-(3-Methoxy-phenyl)-2-oxo-5-phenyl-2,3-dihydro-1H-benzo[e][1,4]diazepin-3-yl]-isobutyramide;
Thiophene-2-carboxylic acid[5-(3-methoxy-phenyl)-2-oxo-5-phenyl-2,3-dihydro-1H-benzo[e][1,4]diazepin-3-yl]-amide;
Cyclohexanecarboxylic acid[5-(3-methoxy-phenyl)-2-oxo-5-phenyl-2,3-dihydro-1H-benzo[e][1,4]diazepin-3-yl]-amide;
Piperidine-1-carboxylic acid[5-(3-methoxy-phenyl)-2-oxo-5-phenyl-2,3-dihydro-1H-benzo[e][1,4]diazepin-3-yl]-amide;
Piperidine-4-carboxylic acid[5-(3-methoxy-phenyl)-2-oxo-5-phenyl-2,3-dihydro-1H-benzo[e][1,4]diazepin-3-yl]-amide;
Cyclohexanecarboxylic acid (8-chloro-2-oxo-5-phenyl-2,3-dihydro-1H-benzo[e][1,4]diazepin-3-yl)-amide;
Thiophene-2-carboxylic acid (8-methyl-2-oxo-5-phenyl-2,3-dihydro-1H-benzo[e][1,4]diazepin-3-yl)-amide;
1-(2-Oxo-5-phenyl-2,3-dihydro-1H-benzo[e][1,4]diazepin-3-yl)-3-thiophene-2-yl-urea;
1-(2-Oxo-5-phenyl-253-dihydro-1H-benzo[e][1,4]diazepin-3-yl)-3-thiophene-3-urea;
Pyridine-2-carboxylic acid (2-oxo-5-phenyl-2,3-dihydro-1H-benzo[e][1,4]diazepin-3-yl)-amide;
1H-Pyrazole-4-carboxylic acid (2-oxo-5-phenyl-2,3-dihydro-1H-benzo[e][1,4]diazepin-3-yl)-amide;
6-Dimethylamino-N-(2-oxo-5-phenyl-2,3-dihydro-1H-benzo[e][1,4]diazepin-3-yl)-nicotinamide;
2-Ethoxy-naphthalene-1-carboxylic acid (2-oxo-5-phenyl-2,3-dihydro-1H-benzo[e][1,4]diazepin-3-yl)-amide;
9-Oxo-9H-fluorene-1-carboxylic acid (2-oxo-5-phenyl-2i(3-dihydro-1H-benzo[e][1,4]diazepin-3-yl)-amide;
2-Oxo-2,3-dihydro-benzoimidazole-1-carboxylic acid (2-oxo-5-phenyl-2,3-dihydro-1H-benzo[e][1,4]diazepin-3-yl)-amide;
(2-Oxo-5-phenyl-2,3-dihydro-1H-benzo[e][1,4]diazepin-3-yl)carbamic acid tert-butyl ester;
4,5-Dibromo-furan-2-carboxylic acid (2-oxo-5-phenyl-2,3-dihydro-1H-benzo[e][1,4]diazepin-3-yl)-amide;
Benzofuran-2-carboxylic acid (2-oxo-5-phenyl-253-dihydro-1H-benzo[e][1,4]diazepin-3-yl)-amide;
(2-Oxo-5-phenyl-2,3-dihydro-1H-benzo[e][1,4]diazepin-3-yl)-carbamic acid methyl ester;
(2-Oxo-5-phenyl-2,3-dihydro-1H-benzo[e][1,4]diazepin-3-yl)-carbamic acid ethyl ester;
(2-Oxo-5-phenyl-2,3-dihydro-1H-benzo[e][1,4]diazepin-3-yl)-carbamic acid isobutyl ester; and
2-Oxo-N-(2-oxo-5-phenyl-2,3-dihydro-1H-benzo[e][1,4]diazepin-3-yl)-2-thiophene-2-yl-acetamide; or
(b) one of the following or an N-oxide thereof:
6-(4-Methyl-piperazin-1-yl)-N-(2-oxo-5-phenyl-2,3-dihydro-1H-benzo[e][1,4]diazepin-3-yl)-nicotinamide;
3,4,5,6-Tetrahydro-2H-[1,21]bipyridinyl-5′-carboxylic acid (2-oxo-5-phenyl-2,3-dihydro-1H-benzo[e][1,4]diazepin-3-yl)-amide;
2-(1,1-Dioxo-1λ6-thiomorpholin-4-yl)-N-(2-oxo-5-phenyl-2,3-dihydro-1H-benzo[e][1,4]diazepin-3-yl-benzamide;
2-Chloro-4-morpholin-4-yl-N-(2-oxo-5-phenyl-2,3-dihydro-1H-benzo[e][1,4]diazepin-3-yl)-benzamide;
2-(1,1-Dioxo-1λ6-thiomorpholin-4-yl)-4-fluoro-(2-oxo-5-phenyl-2,3-dihydro-1H-benzo[e][1,4]diazepin-3-yl-benzamide;
5-Chloro-2-(1,1-dioxo-1λ6-thiomorpholin-4-yl)-N-(2-oxo-5-phenyl-2,3-dihydro-1H-benzo[e][1,4]diazepm-3-yl)-benzamide;
2-(1,1-Dioxo-1λ6-thiomorpholin-4-yl)-5-fluoro-N-(2-oxo-5-phenyl-2,3-dihydro-1H-benzo[e][1,4]diazepin-3-yl)-benzamide;
5-(4-Methyl-piperazin-1-ylmethyl)-furan-2-carboxylic acid (2-oxo-5-phenyl-2,3-dihydro-1H-benzo[e][1,4]diazepin-3-yl)-amide;
5-Pyrrolidin-1-ylmethyl-furan-2-carboxylic acid (2-oxo-5-phenyl-2,3-dihydro-1H-benzo[e][1,4]diazepin-3-yl)-amide;
5-Piperidin-1-ylmethyl-furan-2-carboxylic acid (2-oxo-5-phenyl-2,3-dihydro-1H-benzo[e][1,4]diazepin-3-yl)-amide;
5-Dimethylaminomethyl-furan-2-carboxylic acid (2-oxo-5-phenyl-2,3-dihydro-1H-benzo[e][1,4]diazepin-3-yl)-amide;
4-Fluoro-N-(2-oxo-5-phenyl-253-dihydro-1H-benzo[e][154]diazepin-3-yl)-2-piperidin-1-yl-benzamide;
4-Fluoro-2-morpholino-4-yl-N-(2-oxo-5-phenyl-2,3-dihydro-1H-benzo[e][1,4]diazepin-3-yl)-benzamide;
4-Cyano-N-(2-oxo-5-phenyl-2,3-dihydro-1H-benzo[e][1,4]diazepin-3-yl)-2-pyrrolidin-1-yl-benzamide;
4-Cyano-N-(2-oxo-5-phenyl-253-dihydro-1H-benzo[e][1,4]diazepin-3-yl)-piperidine-1-yl-benzamide;
N-(2-Oxo-5-phenyl-2,3-dihydro-1H-benzo[e][1,4]diazepin-3-yl)-2-pyrrolidin-1-yl4-trifluoromethyl-benzamide;
N-(2-Oxo-5-phenyl-2,3-dihydro-1H-benzo[e][1,4]diazepin-3-yl)-2-piperidin-1-yl-4-trifluoromethyl-benzamide;
2-Morpholin-4-yl-N-(2-oxo-5-phenyl-2,3-dihydro-1H-benzo[e][1,4]diazepin-3-yl)-4-trifluoromethyl-benzamide;
N-(2-Oxo-5-phenyl-2,3-dihydro-1H-benzo[e][1,4]diazepin-3-yl)-2-pyrrolidin-1-yl-5-trifluoromethyl-benzamide;
2-Morpholin-4-yl-N-(2-oxo-5-phenyl-2,3-dihydro-1H-benzo[e][1,4]diazepin-3-yl)-5-trifluoromethyl-benzamide;
2-Morpholin-4-yl-N-(2-oxo-5-phenyl-2,3-dihydro-1H-benzo[e][1,4]diazepin-3-yl)-nicotinamide;
2-(1,1-Dioxo-1λ6-thiomorpholin-4-yl)-N-(2-oxo-5-phenyl-2,3-dihydro-1H-benzo[e][1,4]diazepin-3-yl)-nicotinamide;
2-(1,1-Dioxo-1λ6-thiomorpholin-4-yl)-3-methyl-N-(2-oxo-5-phenyl-2,3-dihydro-1H-benzo[e][1,4]diazepin-3-yl)-benzamide;
2-(1,1-Dioxo-1λ6-thiomorpholin-4-yl)-4-methyl-N-(2-oxo-5-phenyl-2,3-dihydro-1H-benzo[e][1,4]diazepin-3-yl)-benzamide;
2-(1,1-Dioxo-1λ6-thiomorpholin-4-yl)-6-methyl-N-(2-oxo-5-phenyl-2,3-dihydro-1H-benzo[e][1,4]diazepin-3-yl)-benzamide;
2-Chloro-6-(1,1-dioxo-1λ6-thiomorpholin-4-yl)-N-(2-oxo-5-phenyl-2,3-dihydro-1H-benzo[e][1,4]diazepin-3-yl)-benzamide;
3-Cyclopropyl-2-oxo-2,3-dihydro-imidazo[4,5-b]pyridine-1-carboxylic acid (2-oxo-5-phenyl-2,3-dihydro-1H-benzo[e][1,4]diazepin-3-yl)-amide;
3-(4-Methyl-piperazine-1-sulfonyl)-N-(2-oxo-5-phenyl-2,3-dihydro-1H-benzo[e][1,4]diazepin-3-yl)-benzamide;
4-(4-Methyl-piperazin-1-yl)-N-(2-oxo-5-phenyl-2,3-dihydro-1H-benzo[e][1,4]diazepin-3-yl)-benzamide;
N-(2-Oxo-5-phenyl-2,3-dihydro-1H-benzo[e][1,4]diazepin-3-yl)-3-(piperidine-1-sulfonyl)-benzamide;
3-(Morpholine-4-sulfonyl)-N-(2-oxo-5-phenyl-2,3-dihydro-1H-benzo[e][1,4]diazepin-3-yl)-benzamide;
5-Morpholin-4-ylmethyl-furan-2-carboxylic acid (2-oxo-5-phenyl-2,3-dihydro-1H-benzo[e][1,4]diazepin-3-yl)-amide;
5-Hydroxymethyl-furan-2-carboxylic acid (2-oxo-5-phenyl-2,3-dihydro-1H-benzo[e][1,4]diazepin-3-yl)-amide;
5-(1,1-Dioxo-1λ6-thiomorpholin-4-ylmethyl)-furan-2-carboxylic acid (2-oxo-5-phenyl-2,3-dihydro-1H-benzo[e][1,4]diazepin-3-yl)-amide;
2-Chloro-4-(1,1H-dioxo-1λ6-thiomorpholin-4-yl)-N-(2-oxo-5-phenyl-2,3-dihydro-1H-benzo[e][1,4]diazepin-3-yl)-benzamide;
2-Chloro-5-(1,1-dioxo-1λ6-thiomorpholin-4-yl)-N-(2-oxo-5-phenyl-2,3-dihydro-1H-benzo[e][1,4]diazepin-3-yl)-benzamide;
5-{[(2-Methanesulfonyl-ethyl)-methyl-amino]-methyl}-furan-2-carboxylic acid (2-oxo-5-phenyl-2,3-dihydro-1H-benzo[e][1,4]diazepin-3-yl-amide;
2-Pyridin-3-yl-thiazole-4-carboxylic acid (2-oxo-5-phenyl-2,3-dihydro-1H-benzo[e][1,4]diazepin-3-yl)-amide;
2-Pyridin-4-yl-thiazole-4-carboxylic acid (2-oxo-5-phenyl-2,3-dihydro-1H-benzo[e][1,4]diazepin-3-yl)-amide;
4-Methyl-2-pyrazin-2-yl-thiazole-5-carboxylic acid (2-oxo-5-phenyl-2,3-dihydro-1H-benzo[e][1,4]diazepin-3-yl)-amide;
2-Morpholin-4-ylmethyl-furan-3-carboxylic acid (2-oxo-5-phenyl-2,3-dihydro-1H-benzo[e][1,4]diazepin-3-yl)-amide;
3-Morpholin-4-ylmethyl-N-(2-oxo-5-phenyl-2,3-dihydro-1H-benzo[e][1,4]diazepin-3-yl)-benzamide;
5-Morpholin-4-ylmethyl-isoxazole-3-carboxylic acid (2-oxo-5-phenyl-2,3-dihydro-1H-benzo[e][1,4]diazepin-3-yl)-amide;
3-Morpholin-4-ylniethyl-furan-2-carboxylic acid (2-oxo-5-phenyl-2,3-dihydro-1H-benzo[e][1,4]diazepin-3-yl)-amide;
5-Pyridin-2-yl-thiophene-2-carboxylic acid (2-oxo-5-phenyl-2,3-dihydro-1H-benzo[e][1,4]diazepin-3-yl)-amide;
2-Methyl-4-(morpholin-4-sulfonyl)-furan-3-carboxylic acid (2-oxo-5-phenyl-2,3-dihydro-1H-benzo[e][1,4]diazepin-3-yl)-amide;
6-Morpholin-4-yl-N-(2-oxo-5-phenyl-2,3-dihydro-1H-benzo[e][1,4]diazepin-3-yl)-nicotinamide;
3-Morpholin-4-ylmethyl-thiophene-2-carboxylic acid (2-oxo-5-phenyl-2,3-dihydro-1H-benzo[e][1,4]diazepin-3-yl)-amide;
5-Morpholin-4-ylmethyl-thiophene-2-carboxylic acid (2-oxo-5-phenyl-2,3-dihydro-1H-benzo[e][1,4]diazepin-3-yl)-amide;
2-Morpholin-4-yl-N-(2-oxo-5-phenyl-2,3-dihydro-1H-benzo[e][1,4]diazepin-3-yl)-benzamide;
5-Phenyl-oxazole-4-carboxylic acid (2-oxo-5-phenyl-2,3-dihydro-1H-benzo[e][1,4]diazepin-3-yl)-amide; and
1-(2-Oxo-5-phenyl-2,3-dihydro-1H-benzo[e][1,4]diazepin-3-yl)-3-(4-phenoxy-phenyl-)-urea.

9. A process according to claim 8, wherein the benzodiazepine derivative of formula (I) is the S enantiomer.

10. A process according to claim 1 which further comprises formulating the benzodiazepine derivative of formula (I) or a pharmaceutically acceptable salt thereof into a pharmaceutical composition which further comprises a pharmaceutically acceptable carrier or diluent.

11. A compound of formula (II): 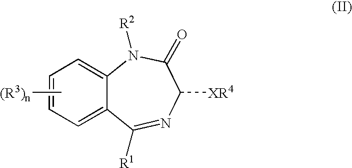
wherein, R¹, R², R³, R⁴, n and X are as defined in claim 1.

12. A compound of formula (IIa): 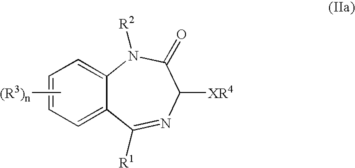
wherein, R¹, R², R³, R⁴, n and X are as defined in claim 1.
